SEQUENCE LISTING (9 sequences)

SEQ ID NO: 1 - protein, 37 aa, Homo sapiens [sequence omitted]

SEQ ID NO: 2 - protein, 37 aa, Artificial Sequence
  Synthetic
KCNTATCATQRLANFLVHSSNNFGPILPPTNVGSNTY

SEQ ID NO: 3 - protein, 32 aa, Homo sapiens
  MISC_FEATURE (32)..(32): amidated at location 32
CGNLSTCMLGTYTQDFNKFHTFPQTAIGVGAP

SEQ ID NO: 4 - protein, 32 aa, Oncorhynchus keta
  MISC_FEATURE (32)..(32): amidated at location 32
CSNLSTCVLGKLSQELHKLQTYPRTNTGSGTP

SEQ ID NO: 5 - protein, 32 aa, Artificial Sequence
  Synthetic
KCNTATCVLGRLSQELHRLQTYPRTNTGSNTY

SEQ ID NO: 6 - protein, 31 aa, Artificial Sequence
  Synthetic
CNTATCVLGRLSQELHRLQTYPRTNTGSNTY

SEQ ID NO: 7 - protein, 32 aa, Artificial Sequence
  Synthetic
KCNTATCALGRLSQELHRLQTYPRTNTGSNTY

SEQ ID NO: 8 - protein, 31 aa, Artificial Sequence
  Synthetic
CNTATCALGRLSQELHRLQTYPRTNTGSNTY

SEQ ID NO: 9 - protein, 32 aa, Artificial Sequence
  Synthetic
HCNTATCALGHLSQELHHLQTYPHTNTGSNTY

(19) United States
(12) Patent Application Publication
(10) Pub. No.: US 2011/0152183 A1
(43) Pub. Date: Jun. 23, 2011

(54) DERIVATISED HYBRID PEPTIDES OF AMYLIN AND SALMON CALCITONIN

(75) Inventors: Lauge Schäffer, Lyngby (DK); Thomas Kruse, Herlev (DK)

(73) Assignee: Novo Nordisk A/S, Bagsvaerd (DK)

(21) Appl. No.: 13/000,225

(22) PCT Filed: Jun. 25, 2009
(86) PCT No.: PCT/EP09/57972

§ 371 Date: Feb. 28, 2011

(30) Foreign Application Priority Data
Jun. 25, 2008 (EP) 08158995.4

(51) Int. Cl. A61K 38/23; C07K 14/585; A61P 3/10; A61P 3/04; A61P 25/28

(52) U.S. Cl. 514/5.3; 530/324; 514/11.9; 514/6.9

(57) ABSTRACT

Described are derivatives of hybrid peptides and pharmaceutical compositions comprising such, wherein said hybrid peptides comprise the C-terminal end of the human amylin peptide sequence, the middle portion of the salmon calcitonin peptide sequence and the N-terminal end of the human amylin peptide sequence, and wherein an albumin binding moiety is attached to the hybrid peptide, optionally via a linker.

### FIELD OF THE INVENTION

The present invention is related to derivatives of hybrid peptides, wherein said hybrid peptides comprise the C-terminal end of the human amylin peptide sequence, the middle portion of the salmon calcitonin peptide sequence and the N-terminal end of the human amylin peptide sequence, and wherein an albumin binding moiety is attached to the hybrid peptide, optionally via a linker.

### BACKGROUND OF THE INVENTION

A large and growing number of people suffer from diabetes mellitus and obesity. Diabetes mellitus is a metabolic disorder in which the ability to utilize glucose is partly or completely lost. The most efficient anti-diabetic agent used to lower blood glucose is insulin and analogue(s) thereof. It has been known for a long time that when traditional insulin is used to treat diabetes, it is associated with an increase in body weight. Human amylin is a 37 amino acid long peptide which has physico-chemical properties that make its use as a drug troublesome. In particular, it has a tendency to fibrillate in-vitro and/or ex-vivo and become ineffective due to precipitation. A drug product called Symlin is currently on the market which contains an analogue of human amylin (pramlintide) where three of the 37 amino acids are substituted to proline. This improves substantially the fibrillating tendency. Pramlintide is known to reduce food intake in humans. However, even pramlintide is difficult to keep in solution at neutral pH and it is therefore provided in an acidic solution i.e. Symlin™.

In mammals, calcitonin functions in the regulation of bone marrow turnover and calcium metabolism. Calcitonin, which is caused to be released from the thyroid by elevated serum calcium levels, produces actions on bone and other organs which tend to reduce serum calcium levels.

Calcitonin has been used clinically for treatment of disorders of calcium metabolism and pain, and its relationship to increased glucose levels in mammals has been the subject of varying reports. The use of calcitonins in the treatment of diabetes mellitus is described in U.S. Pat. No. 5,321,008, issued Jun. 14,1994, and in U.S. Pat. No. 5,508,260, issued Apr. 16,1996.

International patent application WO 2006083254 discloses an amylin family peptide comprising a loop region of amylin and analogs thereof; an [alpha] helix region of at least a portion of an [alpha] helix region of calcitonin or analogs thereof and an [alpha] helix region of a calcitonin or analogs thereof.

### SUMMARY OF THE INVENTION

The present invention is related to derivatives of hybrid peptides, wherein said hybrid peptides comprise the C-terminal end of the human amylin peptide sequence, the middle portion of the salmon calcitonin peptide sequence and the N-terminal end of the human amylin peptide sequence, and wherein an albumin binding moiety is attached to the hybrid peptide, optionally via a linker.

In one aspect derivatives according to the invention are derivatives of analogue hybrid peptides, wherein said analogue hybrid peptides have from 1-12 amino acid substitutions compared to the parent hybrid peptide.

In another aspect the albumin binding moiety of the derivatives according to the invention is attached to the N-terminal amino acid and/or the C-terminal amino acid and/or to one or more amino acids internally in the hybrid peptide.

A derivative according to the invention may comprise an N-terminal extension consisting of 1-12 additional amino acids attached N-terminally to the hybrid peptide, wherein the albumin binding moiety is attached, optionally via a linker, to the N-terminal amino acid of the additional amino acids.

Also derivatives wherein 0-8 additional charges have been added compared to the parent hybrid peptide are contemplated by the invention.

Pharmaceutical compositions comprising a derivative according to the invention and a pharmaceutically acceptable excipients are also described and derivatives according to the invention for use as a medicament.

### DEFINITIONS

The term “potency” is used to describe the effect of a given compound in assays were a sigmoidal relationship between log concentration and the effect of a compound has been established. Furthermore, the response should be variable from 0 to 100%. EC50 (effective concentration 50) can be used to describe the concentration of a given compound yielding a response of 50% in the assay. The potency may be measured in e.g. a Luciferase assay as described in the assay section.

The term “activity” refers in one aspect to the ability to reduce appetite and/or increase satiety. The activity can be measured by the ability to reduce appetite as e.g. described in Pharmacological assays I under the heading ASSAYS.

The term “human amylin” as used herein refers to the peptide human amylin which is co-secreted with insulin from β-cells of the human pancreas and has the sequence as depicted in SEQ ID No1:

SEQ ID No: 1
Lys-Cys-Asn-Thr-Ala-Thr-Cys-Ala-Thr-Gln-Arg-Leu-
Ala-Asn-Phe-Leu-Val-His-Ser-Ser-Asn-Asn-Phe-Gly-
Ala-Ile-Leu-Ser-Ser-Thr-Asn-Val-Gly-Ser-Asn-Thr-
Tyr-NH₂

wherein a disulfide bridge is situated between the cysteins in positions 2 and 7 and the C-terminal end is in the form of an amide group.

With “pramlintide” is herein meant an analogue of human amylin, wherein human amylin has been substituted with three proline residues in the positions 25, 28 and 29 to yield 25Pro,28Pro,29Pro human amylin. Pramlintide thus has the sequence:

SEQ ID No: 2
Lys-Cys-Asn-Thr-Ala-Thr-Cys-Ala-Thr-Gln-Arg-Leu-
Ala-Asn-Phe-Leu-Val-His-Ser-Ser-Asn-Asn-Phe-Gly-
Pro-Ile-Leu-Pro-Pro-Thr-Asn-Val-Gly-Ser-Asn-Thr-
Tyr-NH₂

By “calcitonin” or “CT” is meant the human peptide hormone and species variants thereof, including human calcitonin (h-CT) or salmon calcitonin (sCT).

Calcitonin is a small peptide produced by the parafollicular cells of the thyroid gland in mammals and by the ultimobranchial glands of birds and fish. Many types of Calcitonin have been isolated, such as human Calcitonin, salmon Calcitonin, eel Calcitonin, elkatonin, porcine Calcitonin, and chicken Calcitonin. There is significant structural non-homology among the various Calcitonin types. For example, there is only 50% identity between the amino acids making up human Calcitonin and those making up salmon Calcitonin.

Salmon calcitonin (s-CT) has the sequence consisting of:

SEQ ID No: 4
Cys-Ser-Asn-Leu-Ser-Thr-Cys-Val-Leu-Gly-Lys-Leu-
Ser-Gln-Glu-Leu-His-Lys-Leu-Gln-Thr-Tyr-Pro-Arg-
Thr-Asn-Thr-Gly-Ser-Gly-Thr-Pro-NH₂

Human Calcitonin (h-CT) is a peptide hormone containing 32 amino acid residues which is produced primarily by the Parafollicular (also known as C) cells of the thyroid. Salmon calcitonin is a polypeptide which consists of 32 amino acids. It has a disulphide bridge between the first and seventh amino acids at the amino-terminal end of the polypeptide chain, the disulfide bridge being essential for its biological activity, and a prolinamide group at the carboxyl terminal amino acid. Human calcitonin has the sequence consisting of:

SEQ ID No: 3
Cys-Gly-Asn-Leu-Ser-Thr-Cys-Met-Leu-Gly-Thr-Tyr-
Thr-Gln-Asp-Phe-Asn-Lys-Phe-His-Thr-Phe-Pro-Gln-
Thr-Ala-Ile-Gly-Val-Gly-Ala-Pro-NH₂

The term “parent hybrid peptide” shall herein mean a hybrid peptide comprising the C-terminal end of the human amylin peptide sequence, the middle portion of the salmon calcitonin peptide sequence and the N-terminal end of the human amylin peptide sequence, which has the sequence:

SEQ ID No: 5
Lys-Cys-Asn-Thr-Ala-Thr-Cys-Val-Leu-Gly-Arg-Leu-
Ser-Gln-Glu-Leu-His-Arg-Leu-Gln-Thr-Tyr-Pro-Arg-
Thr-Asn-Thr-Gly-Ser-Asn-Thr-Tyr-NH₂

In the present text, the term “analogue hybrid peptide” or “hybrid analogue” is used to designate a peptide wherein one or more amino acid residues of the parent hybrid peptide independently have been modified i.e. wherein one or more amino acid residues have been substituted by other amino acid residues and/or wherein one or more amino acid residues of the parent hybrid peptide have been deleted and/or wherein one or more amino acid residues have been added to the parent hybrid peptide. In one aspect a substitution or addition is with any natural amino acid.

In one aspect a hybrid analogue comprises less than 17 modifications (substitutions, deletions, additions) relative to the parent hybrid peptide. In one embodiment a hybrid analogue comprises less than 15 modifications (substitutions, deletions, additions) relative to the parent hybrid peptide. In one embodiment a hybrid analogue comprises less than 13 modifications (substitutions, deletions, additions) relative to the parent hybrid peptide. In another embodiment a hybrid analogue comprises less than 11 modifications (substitutions, deletions, additions) relative to the parent hybrid peptide. In another embodiment a hybrid analogue comprises less than 10 modifications (substitutions, deletions, additions) relative to the parent hybrid peptide. In another embodiment a hybrid analogue comprises less than 9 modifications (substitutions, deletions, additions) relative to the parent hybrid peptide. In another embodiment a hybrid analogue comprises less than 8 modifications (substitutions, deletions, additions) relative to the parent hybrid peptide. In one embodiment a hybrid analogue comprises less than 7 modifications (substitutions, deletions, additions) relative to the parent hybrid peptide. In one embodiment a hybrid analogue comprises less than 6 modifications (substitutions, deletions, additions) relative to the parent hybrid peptide. In another embodiment a hybrid analogue comprises less than 5 modifications (substitutions, deletions, additions) relative to the parent hybrid peptide. In another embodiment a hybrid analogue comprises less than 4 modifications (substitutions, deletions, additions) relative to the parent hybrid peptide. In another embodiment a hybrid analogue comprises less than 3 modifications (substitutions, deletions, additions) relative to the parent hybrid peptide. In another embodiment a hybrid analogue comprises less than 2 modifications (substitutions, deletions, additions) relative to the parent hybrid peptide.

Modifications can take place anywhere in the sequence of the hybrid peptide. In one aspect of the invention, modifications of amino acid residues can take place at the N-terminal of the peptide and/or at the C-terminal of the hybrid peptide. In one aspect of the invention the modifications include addition of one or more such as one, two, three or four amino acids in the N-terminal of the hybrid peptide.

Also hybrid analogues, wherein the N-terminal sequence is the N-terminal sequence of salmon calcitonin and the C-terminal sequence is the C-terminal sequence of human amylin are contemplated by the invention as long as said hybrid analogues are within the definition of hybrid analogues as explained above.

The term “hybrid peptide” is herein to be understood as the parent hybrid peptide or a hybrid analogue as defined herein. When two cysteines are present in the N-terminal of the hybrid peptide these should be understood as having a disulfide-bridge. The C-terminal is always in the form of an amide unless otherwise stated.

Hybrid analogues are herein named relative to the sequences of respectively human amylin and salmon calcitonin (s-CT) from which they originate such that the amino acid sequence and numbering of human amylin (Seq ID 1) is used to describe the part(s) of the hybrid analogue derived from human amylin and the amino acid sequence and numbering of salmon calcitonin (Seq ID 4) is used to describe the part(s) of the hybrid analogue derived from s-CT. For example SEQ ID No: 5 as shown above consists of amino acids 1-7 of human amylin, amino acids 8-27 of s-CT wherein amino acids no. 11 and 18 each has been substituted from a lysine residue to an arginine residue, and amino acids 33-37 of human amylin. The name given is thus amylin(1-7)-[Arg11,Arg18]sCT(8-27)-amylin(33-37).

The term “derivative” is used in the present text to designate a peptide in which one or more of the amino acid residues of the peptide have been modified, e.g. by alkylation, acylation, ester formation, amide formation or by maleimide coupling.

The term “a derivative of a hybrid peptide” or “a hybrid peptide derivative” is used in the present text to designate a derivative of the parent hybrid peptide or a hybrid analogue.

The term “derivatised” as used herein means chemically connected via a covalent bond. For example a lysine residue or cysteine residue is linked to an albumin binding residue via a chemical bond. Such a chemical bond can as an example be obtained by derivatisation of an epsilon amino group of lysine by acylation with an active ester of an albumin binding residue such as a long fatty acid. Other examples of derivatisation as used in the present invention includes but is not limited to derivatisation by alkylation, ester formation, amide formation or maleimide coupling.

The term “linker” as used herein means a spacer (the two terms spacer and linker is used interchangeably in the present specification) that separates a peptide and an albumin binding residue. The linker is a chemical moiety which separates the albumin binding moiety and the calcitonin or the analogue thereof by having the linker in between. For example the linker can comprise one or two amino acids which at one end bind to the albumin binding moiety and at the other end binds to the amino acid in position 1 of the hybrid peptide derivative. The chemical moiety of the linker can contribute to/enhance the albumin binding effect of the substituent, e.g. a linker comprising γGlu enhances the albumin binding effect of the hybrid peptide derivative.

The term “albumin binding moiety” as used herein means a residue which binds non-covalently to human serum albumin. The albumin binding residue attached to the therapeutic polypeptide typically has an albumin binding affinity that is below 1 micromolar, preferable below 500 nM and even more preferable below 200 nM or even below 100 nM.

The term “hydrophilic linker” as used herein means a spacer that separates a peptide and an albumin binding residue with a chemical moiety which comprises at least 5 non-hydrogen atoms where 30-50% of these are either N or O.

The term “time of action” refers in the present context to the time span where a pharmacological effect such as reduced food intake is measurable.

The term “stabilized formulation” refers to a formulation with increased physical stability, increased chemical stability or increased physical and chemical stability.

The term “physical stability” of a protein formulation refers as used herein to the tendency of the protein not to form biologically inactive and/or insoluble aggregates of the protein as a result of exposure of the protein to thermo-mechanical stresses and/or interaction with interfaces and surfaces that are destabilizing, such as hydrophobic surfaces and interfaces. Physical stability of the aqueous protein formulations may be evaluated by means of visual inspection, ThT fibrillation assay and/or turbidity measurements as described elsewhere herein. Visual inspection of the formulations is performed in a sharp focused light with a dark background. The turbidity of the formulation is characterized by a visual score ranking the degree of turbidity for instance on a scale from 0 to 3 (a formulation showing no turbidity corresponds to a visual score 0, and a formulation showing visual turbidity in daylight corresponds to visual score 3). A formulation is classified physical unstable with respect to protein aggregation, when it shows visual turbidity in daylight. Alternatively, the turbidity of the formulation can be evaluated by simple turbidity measurements well-known to the skilled person.

The term “chemical stability” of a protein formulation or a pharmaceutical formulation refers to no chemical covalent changes in the protein structure which thereby do not lead to formation of chemical degradation products with potential less biological potency and/or potential increased immunogenic properties compared to the native protein structure. Various chemical degradation products can be formed depending on the type and nature of the native protein and the environment to which the protein is exposed. Elimination of chemical degradation can most probably not be completely avoided and increasing amounts of chemical degradation products is often seen during storage and use of the protein formulation as well-known by the person skilled in the art. Most proteins are prone to deamidation, a process in which the side chain amide group in glutaminyl or asparaginyl residues is hydrolysed to form a free carboxylic acid. Other degradations pathways involves formation of high molecular weight transformation products where two or more protein molecules are covalently bound to each other through transamidation and/or disulfide interactions leading to formation of covalently bound dimer, oligomer and polymer degradation products (pi Stability of Protein Pharmaceuticals, Ahern. T. J. & Manning M. C., Plenum Press, New York 1992). Oxidation (of for instance methionine residues) can be mentioned as another variant of chemical degradation. The chemical stability of the protein formulation can be evaluated by measuring the amount of the chemical degradation products at various time-points after exposure to different environmental conditions (the formation of degradation products can often be accelerated by for instance increasing temperature). The amount of each individual degradation product is often determined by separation of the degradation products depending on molecule size and/or charge using various chromatography techniques (e.g. SEC-HPLC and/or RP-HPLC).

The term “stabilized formulation” refers to a formulation with increased physical stability, increased chemical stability or increased physical and chemical stability compared to an aqueous solution of the peptide.

### DESCRIPTION OF THE INVENTION

The present invention is related to hybrid peptides comprising the C-terminal end of the human amylin peptide sequence, the middle portion of the salmon calcitonin peptide sequence and the N-terminal end of the human amylin peptide sequence.

Hybrid peptides comprising the C-terminal and N-terminal ends of amylin and the middle portion of salmon calcitonin have been shown to have good in vitro and in vivo potency in various amylin models. However; there is still a need to provide derivatives that have the activities of native human amylin, as well as derivatives which have a protracted PK-profile, show enhanced solubility and/or stability over native human amylin.

In one aspect derivatives of hybrid peptides are described which comprise an albumin binding moiety.

It is a particular desire to obtain peptides or derivatives that are stabile when administered non-subcutaneously such as orally, buccally, pulmonary or nasally. In one aspect the hybrid peptide derivatives according to the invention thus have improved enzymatic stability relative to amylin analogues such as pramlintide. The hybrid peptide derivatives according to the invention may thus be particularly useful for the oral, buccal, pulmonary or nasal route of administration.

In one aspect the hybrid peptide derivatives according to the invention have improved stability against enzymatic degradation by trypsin relative to amylin analogues such as pramlintide.

In one aspect a derivative of a hybrid peptide is provided which shows high potency. In a further aspect a derivative of a hybrid peptide is provided which shows improved potency relative to human amylin. In a still further aspect a derivative of a hybrid peptide is provided which shows potency comparable to pramlintide. In a still further aspect a derivative of a hybrid peptide is provided which shows improved potency relative to pramlintide.

In one aspect a derivative of a hybrid peptide is provided which is physical stabile. In another aspect a derivative of a hybrid peptide is provided which has maintained physical stability relative to pramlintide. In a further aspect a derivative of a hybrid peptide is provided which has increased physical stability relative to human amylin.

A hybrid analogue has in one aspect preferably from 20 to 45 naturally occurring or non-naturally occurring amino acids, preferably from 30-35 naturally occurring or non-naturally occurring amino acids.

In one aspect of the invention, the amino acid sequence of the hybrid peptide derivative is selected from the group consisting of:

amylin(1-7)-[Arg11,Arg18]sCT(8-27)-amylin(33-37) (seq ID No: 5)

amylin(2-7)-[Arg11,Arg18]sCT(8-27)-amylin(33-37) (seq ID No: 6)

amylin(1-8)-[Arg11,Arg18]sCT(9-27)-amylin(33-37) (seq ID No: 7)

amylin(2-8)-[Arg11,Arg18]sCT(9-27)-amylin(33-37) (seq ID No: 8)

[His1]amylin(1-8)-[His11,His18,His24]sCT(9-27)-amylin (33-37) (seq ID No: 9)

wherein at least one of the amino acid residue(s) is linked to an albumin binding residue, optionally via a linker, wherein, when two cysteines are present in the N-terminal of the hybrid peptide, a disulfide-bridge is bridging said two cysteines, and wherein the C-terminal is in the form of an amide unless otherwise stated.

The peptides of the invention are generally C-terminally amidated when expressed physiologically, but need not be for the purposes of the instant invention and may thus have a free —OH or —NH₂ group or other posttranslational modifications.

In one aspect substitution or addition of amino acid residues comprises substitution and/or addition of glutamic acid residue(s), lysine residue(s), arginine residue(s), histidine residue(s) and/or aspartic acid residue(s) to obtain a hybrid analogue having 0-8 additional charges compared to the parent hybrid peptide. In a further aspect substitution or addition of amino acid residues comprises substitution and/or addition of histamine residue(s) and/or arginine residue(s) to obtain a hybrid analogue having 0-8 additional charges compared to the parent hybrid peptide. In a still further aspect substitution or addition of amino acid residues comprises substitution and/or addition of histidine residue(s) to obtain a hybrid analogue having 0-8 additional charges compared to the parent hybrid peptide.

Any amino acid position in the hybrid peptide may be derivatised. In one aspect of the invention, the amino acid residue which is derivatised comprises an amino group. Examples of amino acid residues comprising an amino group is lysine, ornithine, Epsilon-N-alkylated lysine such as Epsilon-N methylLysine, O-aminoethylserine, O-aminopropylserine or longer O-alkylated serines containing a primary or secondary amino group in the side chain. In a further aspect of the invention, the derivatised amino acid residue comprises a primary amino group in a side chain. Examples of amino acid residues comprising a primary amino group is lysine, ornithine, O-aminoethylserine, O-aminopropylserine or longer O-alkylated serines containing a primary amino group in the side chain. In yet a further aspect of the invention, the derivatised amino acid residue is lysine. In yet a further aspect of the invention, the derivative according to the invention is only derivatised in one position, e.g. only one amino acid residue is derivatised.

In one aspect of the invention, the linker comprises one or more alkylene glycol units, such as 1 to 5 alkylene glycol units. The alkylene glycol units are in a further aspect ethylene glycol, propylene glycol or butylene glycol but can also be higher alkylene glycols.

In another aspect of the invention, the linker is a hydrophilic linker selected from

—(CH₂)ₗD[(CH₂)ₙE]ₘ(CH₂)ₚ—Q_{q}—, wherein

l, m and n independently are 1-20 and p is 0-10,

Q is —Z—(CH₂)ₗD[(CH₂)ₙG]ₘ(CH₂)ₚ—,

q is an integer in the range from 0 to 5,

each D, E, and G are independently selected from —O—, —NR³—, —N(COR⁴)—, —PR⁵(O)—, and —P(OR⁶)(O)—, wherein R³, R⁴, R⁵, and R⁶ independently represent hydrogen or C₁₋₆-alkyl,

Z is selected from —C(O)NH—, —C(O)NHCH₂—, —OC(O)NH—, C(O)NHCH₂CH₂—, —C(O)CH₂—, —C(O)CH═CH—, —(CH₂)ₛ—, —C(O)—, —C(O)O— or —NHC(O)—, wherein s is 0 or 1.

In another aspect of the invention, the linker is a hydrophilic linker as defined above wherein l is 1 or 2, n and m are independently 1-10 and p is 0-10.

In another aspect of the invention, the linker is a hydrophilic linker as defined above wherein D is —O—.

In another aspect of the invention, the linker is a hydrophilic linker as defined above wherein E is —O—.

In yet another aspect of the invention, the hydrophilic linker is

—CH₂O[(CH₂)₂O]ₘ(CH₂)ₚQ_{q}—, wherein m is 1-10, p is 1-3, and Q is —Z—CH₂O[(CH₂)₂O]ₘ(CH₂)ₚ wherein Z is as defined above.

In another aspect of the invention, the linker is a hydrophilic linker as defined above wherein q is 1.

In another aspect of the invention, the linker is a hydrophilic linker as defined above wherein G is —O—.

In another aspect of the invention, the linker is a hydrophilic linker as defined above wherein Z is selected from the group consisting of —C(O)NH—, —C(O)NHCH₂—, and —OC(O)NH—.

In another aspect of the invention, the linker is a hydrophilic linker as defined above wherein q is 0.

In another aspect of the invention, the linker is a hydrophilic linker as defined above wherein l is 2.

In another aspect of the invention, the linker is a hydrophilic linker as defined above wherein n is 2.

In one aspect of this invention a “hydrophilic linker” is used that separates a peptide and an albumin binding residue with a chemical moiety.

In one aspect of this invention, the hydrophilic linker is —C(O)—(CH₂)ₗ—O—[(CH₂CH₂—O]ₘ—(CH₂)ₚ—[NHC(O)—(CH₂)ₗ—O—[(CH₂)ₙ—O]ₘ(CH₂)ₚ]_{q}—NH—, wherein l, m, n, and p independently are 1-5, and q is 0-5.

In yet another aspect of this invention, the hydrophilic linker is —C(O)—CH₂—O—CH₂CH₂—O—CH₂CH₂[NHC(O)—CH₂—O—CH₂CH₂O —CH₂CH₂]_{q}—NH—, wherein q is 0-5.

In yet another aspect of this invention, the hydrophilic linker is —C(O)—CH₂—O—CH₂CH₂—O—CH₂CH₂—NHC(O)—CH₂—O—CH₂CH₂O —CH₂CH₂—NH—.

In yet another aspect of the invention, the hydrophilic linker is —[CH₂CH₂O]ₘ₊₁(CH₂)ₚQ_{q}- wherein m and p independently is 0-10, and

Q is —Z—(CH₂)ₗD[(CH₂)ₙG]ₘ(CH₂)ₚ- as defined above.

In yet another aspect of the invention, the hydrophilic linker is —(CH₂)ₗ—O—[(CH₂)ₙ—O]ₘ—(CH₂)ₚ—[C(O)NH—(CH₂)ₗ—O—[(CH₂)ₙ—O]ₘ—(CH₂)ₚ]_{q}—, wherein l, m, n, and p independently are 1-5, and q is 0-5.

In a further aspect of the invention, the linker comprises an amino acid residue except Cys, or a dipeptide such as Gly-Lys. In the present text, the expression “a dipeptide such as Gly-Lys” is used to designate a dipeptide wherein the C-terminal amino acid residue is Lys, His or Trp, preferably Lys, and wherein the N-terminal amino acid residue is selected from the group comprising Ala, Arg, Asp, Asn, Gly, Glu, Gln, Ile, Leu, Val, Phe and Pro.]

In one aspect a hybrid peptide according to the invention is acylated with any albumin binder.

In a further aspect, only one lysine residue in a hybrid peptide derivative is linked to an albumin binding residue, optionally via a linker.

In one aspect, the hybrid peptide according to the invention comprises a hydrophilic spacer between the hybrid peptide and one or more albumin binding residue(s).

In one aspect, the hydrophilic spacer is an unbranched oligo ethylene glycol moiety with appropriate functional groups at both terminals that forms a bridge between an amino group of the hybrid peptide and a functional group of the albumin binding residue.

A range of albumin binding residues are known among linear and branched lipohophillic moieties containing 4-40 carbon atoms having a distal acidic group.

In one aspect of the invention, the albumin binding residue is a lipophilic residue. In a further aspect, the lipophilic residue is attached to a lysine residue optionally via a linker by conjugation chemistry such as by alkylation, acylation, ester formation, or amide formation or to a cysteine residue by maleimide coupling.

In a further aspect of the invention, the albumin binding residue is negatively charged at physiological pH. In another aspect of the invention, the albumin binding residue comprises a group which can be negatively charged. One preferred group which can be negatively charged is a carboxylic acid group.

In yet another aspect of the invention, the albumin binding residue is selected from the group consisting of a straight chain alkyl group, a branched alkyl group, a group which has an ω-carboxylic acid group, and a partially or completely hydrogenated cyclopentanophenanthrene skeleton.

In a further aspect of the invention, the albumin binding residue is a cibacronyl residue.

In a further aspect of the invention, the albumin binding residue has from 6 to 40 carbon atoms, from 8 to 26 carbon atoms or from 8 to 20 carbon atoms.

In a further aspect of the invention, the albumin binding residue is an acyl group selected from the group comprising CH₃(CH₂)ᵣCO—, wherein r is an integer from 4 to 38, preferably an integer from 4 to 24, more preferred selected from the group comprising CH₃(CH₂)₆CO—, CH₃(CH₂)₈CO—, CH₃(CH₂)₁₀CO—, CH₃(CH₂)₁₂CO—, CH₃(CH₂)₁₄CO—, CH₃(CH₂)₁₆CO—, CH₃(CH₂)₁₈CO—, CH₃(CH₂)₂₀CO— and CH₃(CH₂)₂₂CO—.

In another aspect of the invention, the albumin binding residue is an acyl group of a straight-chain or branched alkane α,ω-dicarboxylic acid.

In one aspect of the invention, at least one amino acid residue is derivatised with A-B-C-D-

wherein A- is selected from the group consisting of

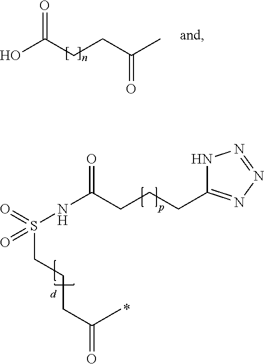

wherein n is selected from the group consisting of 14, 15, 16 17, 18 and 19, p is selected from the group consisting of 10, 11, 12, 13 and 14, and d is selected from the group consisting of 0, 1, 2, 3, 4 and 5,

—B— is selected from the group consisting of

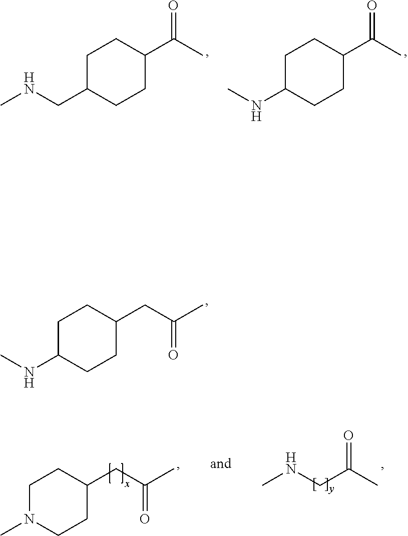

wherein x is selected from the group consisting of 0, 1, 2, 3 and 4, and y is selected from the group consisting of 1, 2, 3, 4, 5, 6, 7, 8, 9, 10, 11 and 12,

—C— is selected from the group consisting of

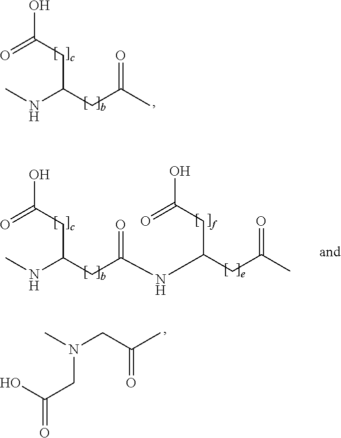

wherein b and e are each independently selected from the group consisting of 0, 1 and 2, and c and f are each independently selected from the group consisting of 0, 1 and 2 with the proviso that b is 1 or 2 when c is 0, or b is 0 when c is 1 or 2, and e is 1 or 2 when f is 0, or e is 0 when f is 1 or 2, and

-D- is attached to said amino acid residue and is a linker.

In one aspect, one amino acid residue is derivatised with A-B-C-D-.

In one aspect of the invention, the derivatised amino acid residue comprises an amino group.

In one aspect of the invention, the derivatised amino acid residue comprises a primary amino group in a side chain.

In one aspect of the invention, the derivatised amino acid residue is lysine.

In one aspect of the invention, A- is

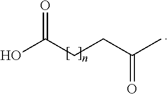

In one aspect of the invention, n is selected from the group consisting of 15 and 17, and more preferred is 17.

In one aspect of the invention, A- is

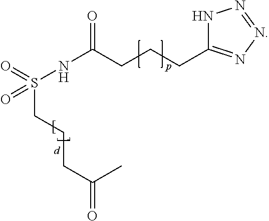

In one aspect of the invention, p is selected from the group consisting of 12, 13, and 14 and more preferred is 13. In a further aspect of the invention, d is selected from the group consisting of 0, 1, 2, 3 and 4, more preferred 0, 1 and 2 and most preferred 1. In a further aspect of the invention, d is selected from the group consisting of 0, 1 and 2 and p is selected from the group consisting of 12, 13 or 14, more preferred d is selected from the group consisting of 1 and 2 and p is selected from the group consisting of 13 and 14, and most preferred d is 1 and p is 13.

In a one aspect of the invention, —B— is

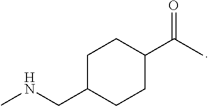

In a further aspect of the invention, —B— is

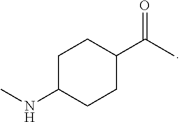

In a further aspect of the invention, —B— is

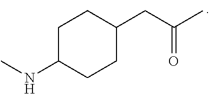

In a further aspect of the invention, —B— is

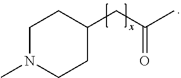

In a further aspect of the invention, x is selected from the group consisting of 0, 1 and 2, more preferred x is selected from the group consisting of 0 and 1 and most preferred x is 1.

In a further aspect of the invention, —B— is

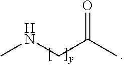

In a further aspect of the invention, y is selected from the group consisting of 2, 3, 4, 5, 6, 7, 8, 9 and 10 and more preferred y is selected from the group consisting of 2, 3, 4, 5, 6, 7, and 8.

In a further aspect of the invention, —C— is

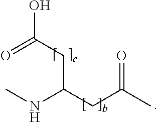

In a further aspect of the invention, c is selected from the group consisting of 0 and 1 and b is selected from the group consisting of 1 and 2, more preferred b is 1 and c is 0.

In a further aspect of the invention, —C— is

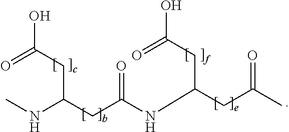

In a further aspect of the invention, is selected from the group consisting of 0 and 1 and e is selected from the group consisting of 1 and 2, more preferred e is 1 and f is 0.

In a further aspect of the invention, —C— is

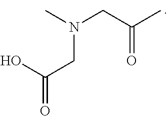

In a further aspect of the invention, D is selected from the group consisting of

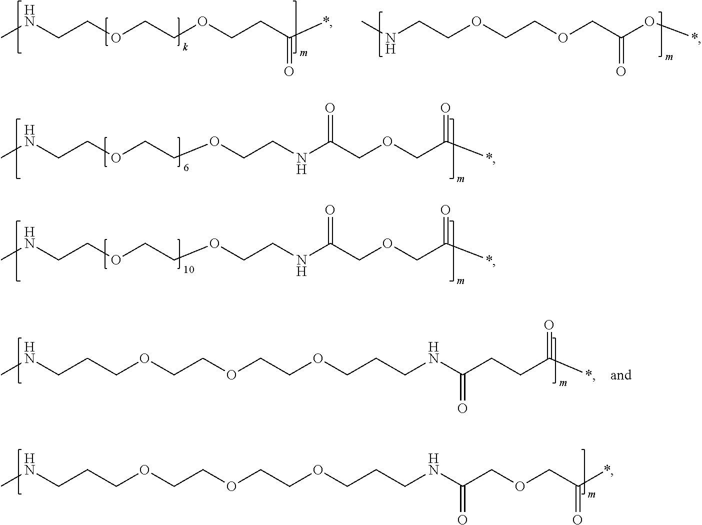

wherein k is selected from the group consisting of 0, 1, 2, 3, 4, 5, 11 and 27, and m is selected from the group consisting of 0, 1, 2, 3, 4, 5 and 6.

In a further aspect of the invention, -D- is

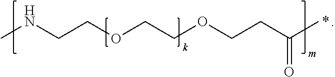

In a further aspect of the invention, k is selected from the group consisting of 1, 2, 3, 11 and 27 and more preferred k is 1.

In a further aspect of the invention, m is selected from the group consisting of 0, 1, 2, 3, and 4 and more preferred m is selected from the group consisting of 0, 1 and 2.

In a further aspect of the invention, -D- is

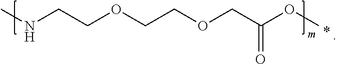

In a further aspect of the invention, m is selected from the group consisting of 0, 1, 2, 3, and 4 and more preferred m is selected from the group consisting of 0, 1 and 2.

In a further aspect of the invention, -D- is

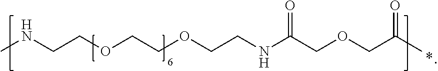

In a further aspect of the invention, m is selected from the group consisting of 0, 1, 2, 3, and 4 and more preferred m is selected from the group consisting of 0, 1 and 2.

In a further aspect of the invention, -D- is

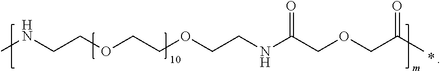

In a further aspect of the invention, m is selected from the group consisting of 0, 1, 2, 3, and 4 and more preferred m is selected from the group consisting of 0, 1 and 2.

In a further aspect of the invention, -D- is

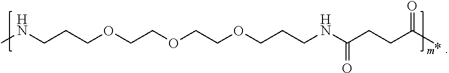

In a further aspect of the invention, m is selected from the group consisting of 0, 1, 2, 3, and 4 and more preferred m is selected from the group consisting of 0, 1 and 2.

In a further aspect of the invention, -D- is

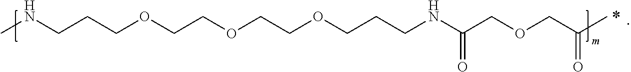

In a further aspect of the invention, m is selected from the group consisting of 0, 1, 2, 3, and 4 and more preferred m is selected from the group consisting of 0, 1 and 2.

In a further aspect of the invention, A-B-C-D- is selected and combined from

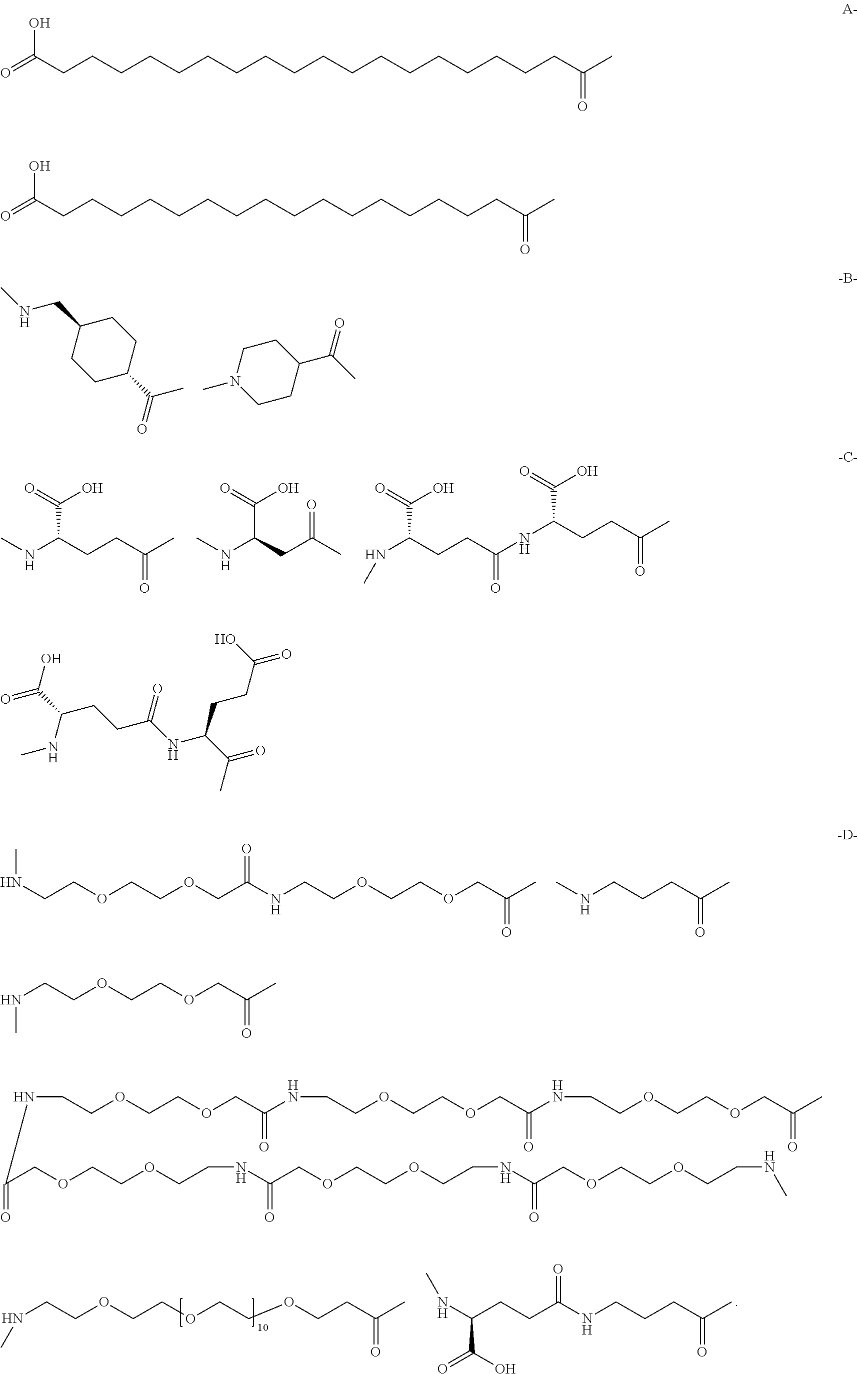

In a further aspect of the invention, A-B-C-D- is selected and combined from

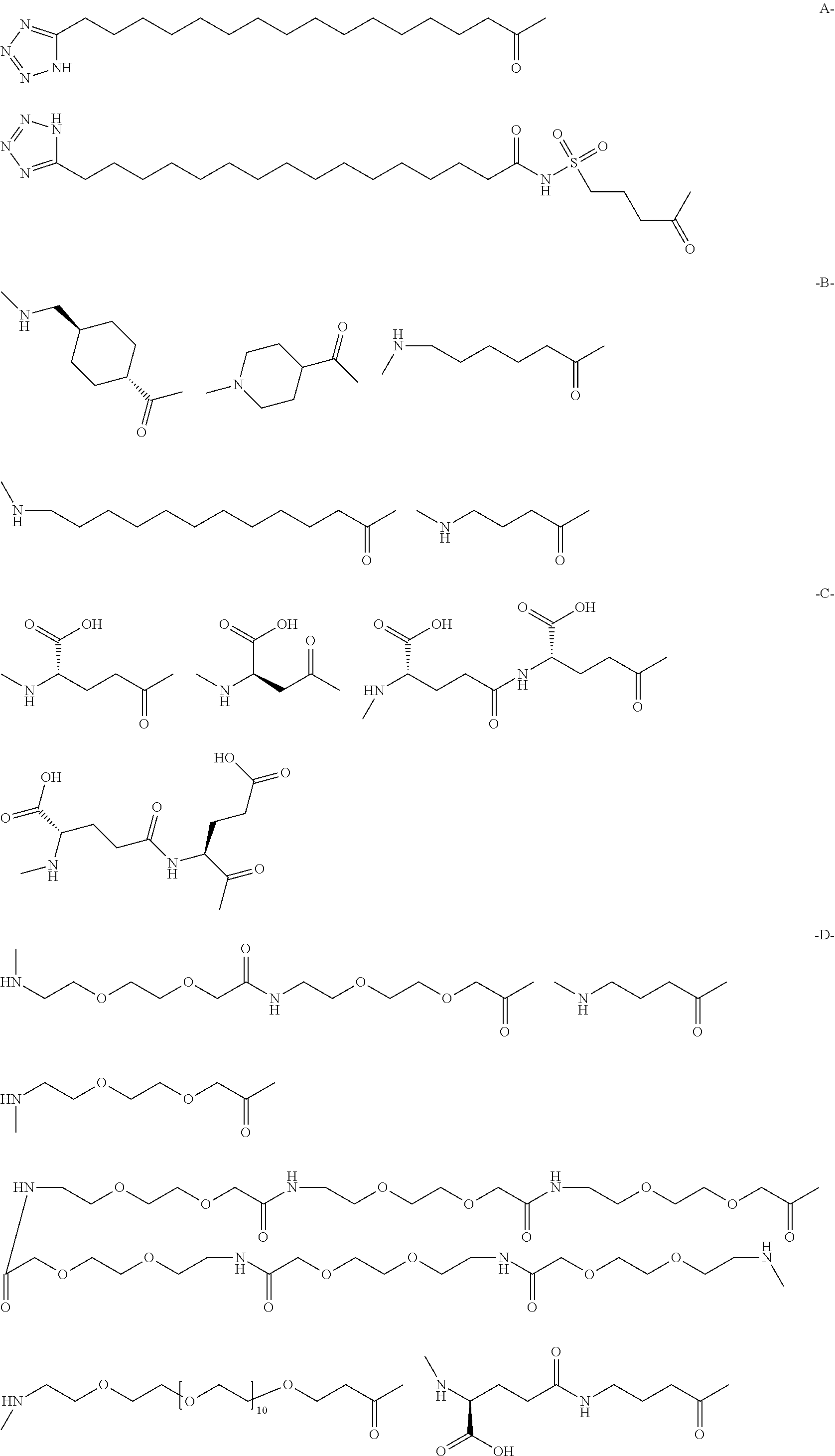

In a further aspect of the invention, A-B-C-D- is selected from the group consisting of

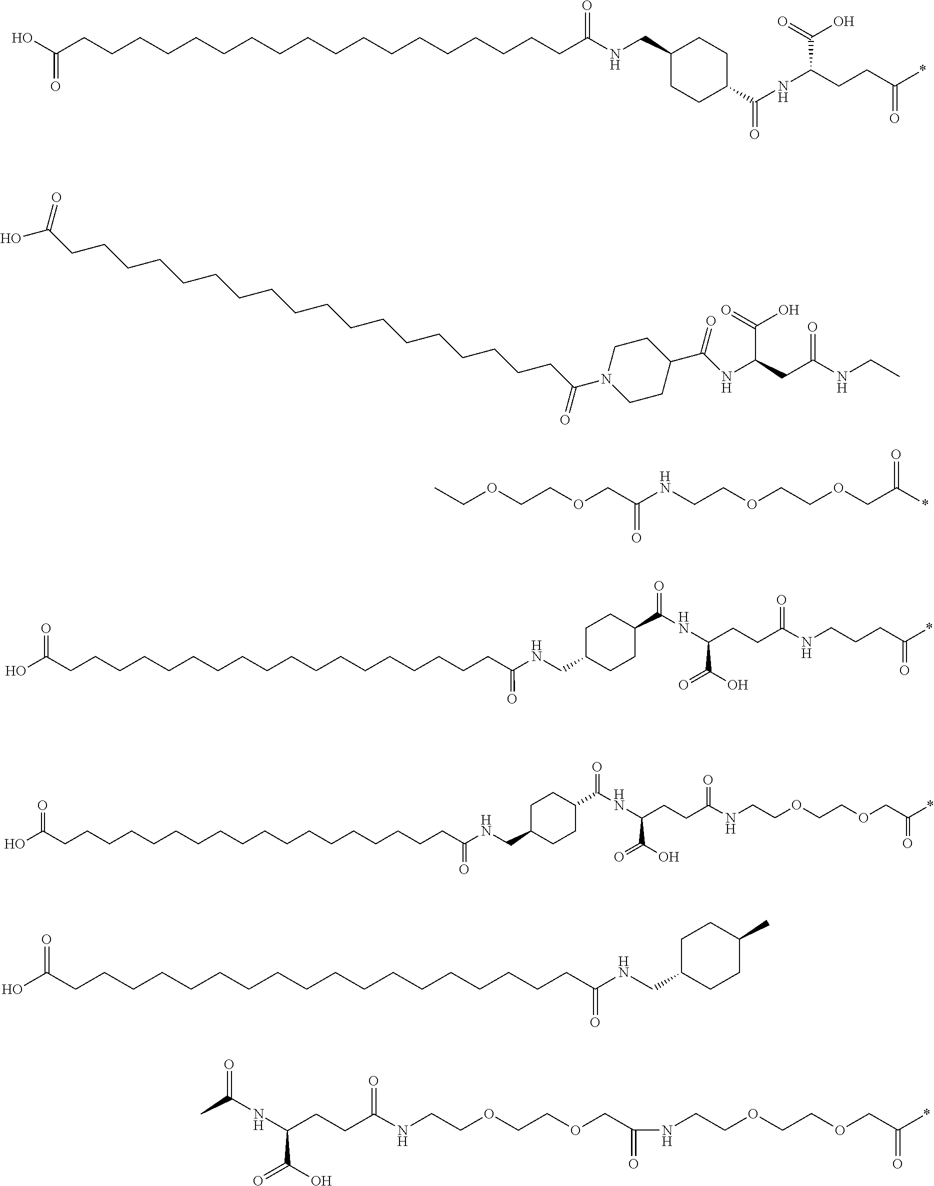

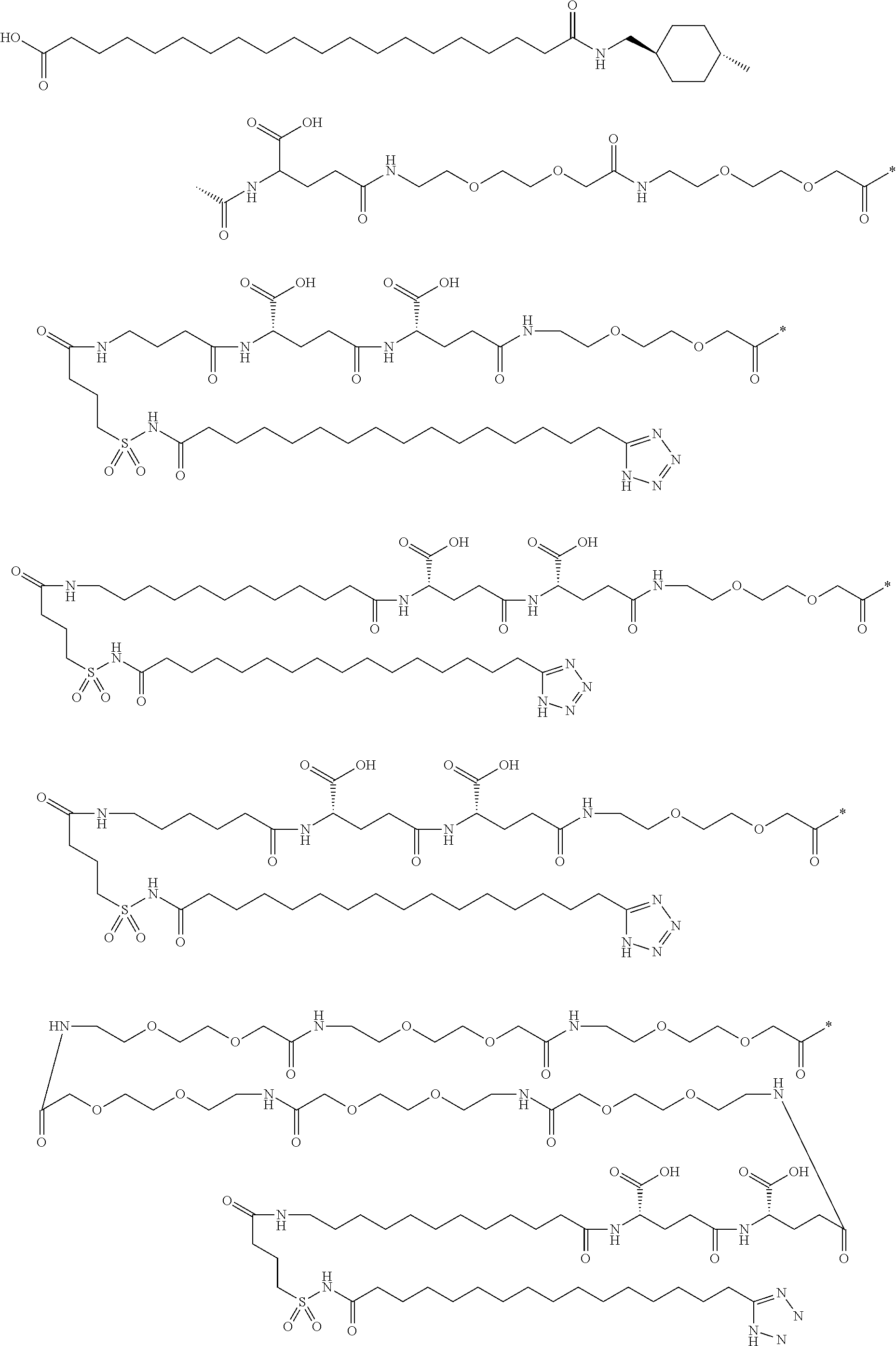

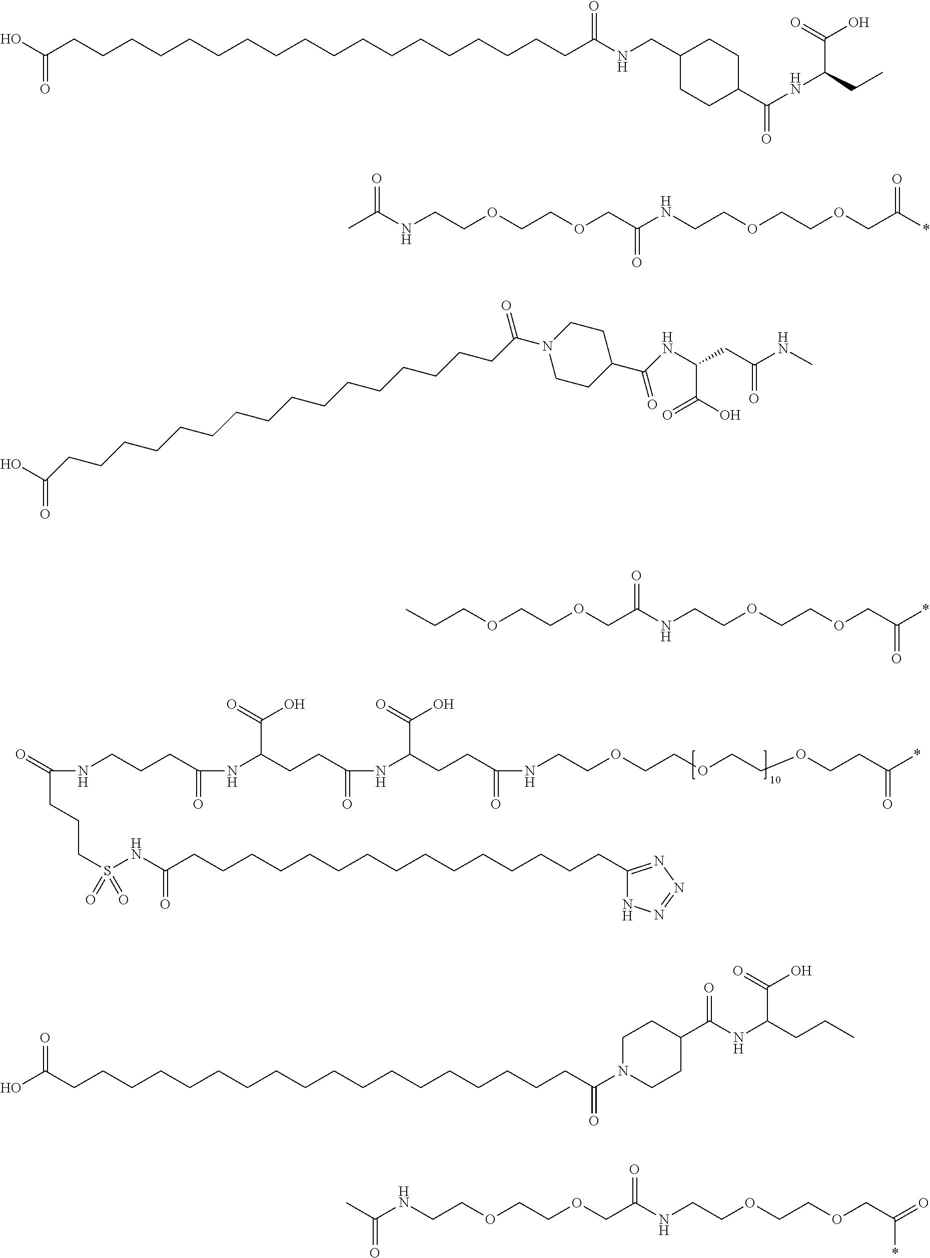

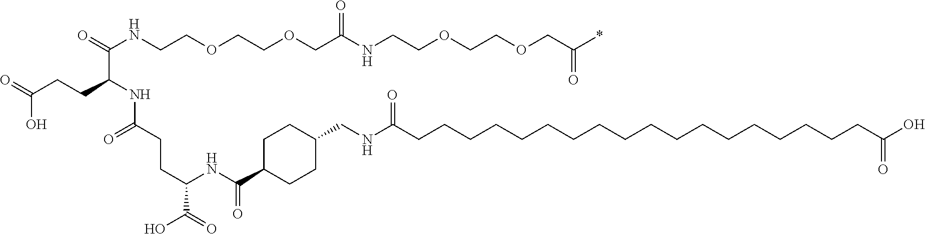

In one aspect a hybrid peptide according to the invention is acylated in the epsilon amino group of Lys1 and/or in the epsilon amino group of a Lysine which has been introduced by substitution into one or more positions of the hybrid peptide and/or in the N-terminal alpha amino group of the full-length or truncated hybrid peptide. In another aspect a hybrid peptide according to the invention is acylated in the epsilon amino group of Lys1 of the full-length or truncated hybrid peptide. In another aspect a hybrid peptide according to the invention is acylated in the epsilon amino group of a Lysine which has been introduced by substitution into one or more positions of the full-length or truncated hybrid peptide. In another aspect a hybrid peptide according to the invention is acylated in the N-terminal alpha amino group of the full-length or truncated hybrid peptide.

In a further aspect a hybrid peptide is acylated with an albumin binder which is C20diacid-gammaGlu or C18diacid-gammaGlu.

In one aspect according to the invention, the hybrid peptide derivative has a protracted pharmaco-kinetic profile compared to human amylin or pramlintide as measured by standard procedures such as ELISA known to people skilled in the art. The pharmaco-kinetic profile may be measured as the half-life, T½, of the hybrid peptide derivative. In one embodiment of the invention T½ is increased relative to human amylin. In a further embodiment T½ is increased relative to pramlintide. In a yet further embodiment T½ is increased at least 2-fold relative to pramlintide. In a yet further embodiment T½ is increased at least 3-fold relative to pramlintide. In a yet further embodiment T½ is increased at least 4-fold relative to pramlintide. In a yet further embodiment T½ is increased at least 5-fold relative to pramlintide. In a yet further embodiment T½ is increased at least 10-fold relative to pramlintide.

In one aspect a hybrid derivative according to the invention has a plasma half-life which is at least 25 hours. In another aspect a hybrid derivative according to the invention has a plasma half-life which is at least 50 hours. In another aspect a hybrid derivative according to the invention has a plasma half-life which is at least 75 hours. In yet another aspect a hybrid derivative according to the invention has a plasma half-life which is at least 100 hours.

The ability of a hybrid peptide derivative according to the invention to reduce cumulative food intake may be measured according to the Pharmacological Assay (I) described in the ASSAY section of the patent application. In one aspect according to the invention, the hybrid peptide derivatives of the present invention may demonstrate an ability to reduce cumulative food intake more than 5% when administering 30 nmol hybrid peptide derivative per kg body-weight compared to administration of the vehicle, preferably more than 15%, more preferably more than 25%, even more preferably more than 35% or 40% most preferably more than 50% over the vehicle.

In one aspect according to the invention, the hybrid peptide derivatives of the present invention may demonstrate an ability to reduce cumulative food intake more than 15% over administration of the vehicle within the first 24 hours after administration, preferably more than 25%, more preferably more than 35%, even more preferably more than 45% or 55% most preferably more than 60% within the first 24 hours after administration.

In one aspect according to the invention, the hybrid peptide derivatives of the present invention may demonstrate an ability to reduce cumulative food intake more than 5% over administration of the vehicle from 24-48 hours after administration, preferably more than 15%, more preferably more than 25%, even more preferably more than 30% when measured for the period from 24-48 hours after administration.

In one aspect according to the invention, the hybrid peptide derivative is less likely to fibrillate, gelate and/or aggregate in vivo and/or ex-vivo compared to human amylin. The tendency of fibrillation and/or gelation and/or aggregate formation may e.g. be estimated in a Thioflavin T test which is known to the person skilled in the art and further described pharmaceutical compositions section herein. Alternatively physical stability may be evaluated by means of visual inspection and/or turbidity measurements as also known to the skilled person.

The production of peptides such as hybrid peptides is well known in the art. The peptides of the invention can thus be produced by classical peptide synthesis, e.g. solid phase peptide synthesis using t-Boc or Fmoc chemistry or other well established techniques, see e.g. Greene and Wuts, “Protective Groups in Organic Synthesis”, John Wiley & Sons, 1999. The peptides may also be produced by a method which comprises culturing a host cell containing a DNA sequence encoding the polypeptide and capable of expressing the polypeptide in a suitable nutrient medium under conditions permitting the expression of the peptide. For peptides comprising non-natural amino acid residues, the recombinant cell should be modified such that the non-natural amino acids are incorporated into the peptide, for instance by use of tRNA mutants.

### Pharmaceutical Compositions

Pharmaceutical compositions containing a hybrid peptide derivative according to the present invention may be prepared by conventional techniques, e.g. as described in Remington's Pharmaceutical Sciences, 1985 or in Remington: The Science and Practice of Pharmacy, 19^{th} edition, 1995.

Another object of the present invention is to provide a pharmaceutical formulation comprising an hybrid analogue according to the present invention which is present in a concentration from 0.1 mg/ml to 500 mg/ml, and wherein said formulation has a pH from 2.0 to 10.0. The formulation may further comprise protease inhibitor(s), a buffer system, preservative(s), tonicity agent(s), chelating agent(s), stabilizers and surfactants.

In one aspect of the invention the pharmaceutical formulation is an aqueous formulation, i.e. formulation comprising water. Such formulation is typically a solution or a suspension. In a further aspect of the invention the pharmaceutical formulation is an aqueous solution. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water.

One object of the present invention is to provide a pharmaceutical formulation comprising a hybrid peptide according to the present invention. In one aspect, the peptide is present in the formulation at a concentration of from about 0.1 mg/ml to about 25 mg/ml. In another aspect, the peptide is present in the formulation at a concentration of from about 1 mg/ml to about 10 mg/ml.

In another aspect, the formulation has a pH from 4.0 to 10.0.

In another aspect, the formulation has a pH from 4.0 to 8.5.

In yet another aspect, the formulation has a pH from 4.0 to 8.0.

In yet another aspect, the formulation has a pH which is 4.0.

In yet another aspect, the formulation has a pH from7.0 to 8.0.

The formulation may further comprise a buffer system, preservative(s), isotonicity agent(s), chelating agent(s), stabilizers and/or surfactants. The use of such excipients in pharmaceutical compositions is well-known to the skilled person. For convenience reference is made to Remington: The Science and Practice of Pharmacy, 19^{th} edition, 1995.

In another aspect the pharmaceutical formulation is a freeze-dried formulation, whereto the physician or the patient adds solvents and/or diluents prior to use.

In another aspect the pharmaceutical formulation is a dried formulation (e.g. freeze-dried or spray-dried) ready for use without any prior dissolution.

In a further aspect of the invention the buffer is selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethan, bicine, tricine, malic acid, succinate, maleic acid, fumaric acid, tartaric acid, aspartic acid or mixtures thereof. Each one of these specific buffers constitutes an alternative aspect of the invention.

In a further aspect of the invention the formulation further comprises a pharmaceutically acceptable preservative. In a further aspect of the invention the formulation further comprises an isotonic agent. In a further aspect of the invention the formulation further comprises a chelating agent.

In a further aspect of the invention the formulation further comprises a stabilizer. The use of a stabilizer in pharmaceutical compositions is well-known to the skilled person. For convenience reference is made to Remington: The Science and Practice of Pharmacy, 19^{th} edition, 1995.

More particularly, compositions of the invention are stabilized liquid pharmaceutical compositions whose therapeutically active components include a polypeptide that possibly exhibits aggregate formation during storage in liquid pharmaceutical formulations. By “aggregate formation” is intended a physical interaction between the peptides that results in formation of oligomers, which may remain soluble, or large visible aggregates that precipitate from the solution. By “during storage” is intended a liquid pharmaceutical composition or formulation once prepared, is not immediately administered to a subject. Rather, following preparation, it is packaged for storage, either in a liquid form, in a frozen state, or in a dried form for later reconstitution into a liquid form or other form suitable for administration to a subject. By “dried form” is intended the liquid pharmaceutical composition or formulation is dried either by freeze drying (i.e., lyophilization; see, for example, Williams and Polli (1984) J. Parenteral

Sci. Technol. 38:48-59), spray drying (see Masters (1991) in Spray-Drying Handbook (5th ed; Longman Scientific and Technical, Essez, U.K.), pp. 491-676; Broadhead et al. (1992) Drug Devel. Ind. Pharm. 18:1169-1206; and Mumenthaler et al. (1994) Pharm. Res. 11:12-20), or air drying (Carpenter and Crowe (1988) Cryobiology 25:459-470; and Roser (1991) Biopharm. 4:47-53). Aggregate formation by a polypeptide during storage of a liquid pharmaceutical composition can adversely affect biological activity of that polypeptide, resulting in loss of therapeutic efficacy of the pharmaceutical composition. Furthermore, aggregate formation may cause other problems such as blockage of tubing, membranes, or pumps when the polypeptide-containing pharmaceutical composition is administered using an infusion system.

The pharmaceutical compositions of the invention may further comprise an amount of an amino acid base sufficient to decrease aggregate formation by the polypeptide during storage of the composition. By “amino acid base” is intended an amino acid or a combination of amino acids, where any given amino acid is present either in its free base form or in its salt form. Where a combination of amino acids is used, all of the amino acids may be present in their free base forms, all may be present in their salt forms, or some may be present in their free base forms while others are present in their salt forms. In one aspect, amino acids to use in preparing the compositions of the invention are those carrying a charged side chain, such as arginine, lysine, aspartic acid, and glutamic acid. Any stereoisomer (i.e., L, D, or a mixture thereof) of a particular amino acid (e.g. methionine, histidine, imidazole, arginine, lysine, isoleucine, aspartic acid, tryptophan, threonine and mixtures thereof) or combinations of these stereoisomers, may be present in the pharmaceutical compositions of the invention so long as the particular amino acid is present either in its free base form or its salt form. In one aspect the L-stereoisomer is used. Compositions of the invention may also be formulated with analogues of these amino acids. By “amino acid analogue” is intended a derivative of the naturally occurring amino acid that brings about the desired effect of decreasing aggregate formation by the polypeptide during storage of the liquid pharmaceutical compositions of the invention. Suitable arginine analogues include, for example, aminoguanidine, ornithine and N-monoethyl L-arginine, suitable methionine analogues include ethionine and buthionine and suitable cysteine analogues include S-methyl-L cysteine. As with the other amino acids, the amino acid analogues are incorporated into the compositions in either their free base form or their salt form. In a further aspect of the invention the amino acids or amino acid analogues are used in a concentration, which is sufficient to prevent or delay aggregation of the protein.

The pharmaceutical compositions may also comprise additional stabilizing agents, which further enhance stability of a therapeutically active polypeptide therein. The use of a stabilizing agents in pharmaceutical compositions is well-known to the skilled person. For convenience reference is made to Remington: The Science and Practice of Pharmacy, 19^{th} edition, 1995.

In a further aspect of the invention the formulation further comprises a surfactant. In a further aspect of the invention the formulation further comprises protease inhibitors. The use of a protease inhibitor is particular useful in pharmaceutical compositions comprising zymogens of proteases in order to inhibit autocatalysis.

In a further embodiment of the invention the formulation further comprises a surfactant. The term “surfactant” as used herein refers to any peptides or ions that are comprised of a water-soluble (hydrophilic) part, the head, and a fat-soluble (lipophilic) segment. Surfactants accumulate preferably at interfaces, which the hydrophilic part is orientated towards the water (hydrophilic phase) and the lipophilic part towards the oil- or hydrophobic phase (i.e. glass, air, oil etc.). The concentration at which surfactants begin to form micelles is known as the critical micelle concentration or CMC. Furthermore, surfactants lower the surface tension of a liquid. Surfactants are also known as amphipathic compounds. The term “Detergent” is a synonym used for surfactants in general. The use of a surfactant in pharmaceutical compositions is well-known to the skilled person. For convenience reference is made to Remington: The Science and Practice of Pharmacy, 19^{th} edition, 1995.

In a further embodiment of the invention the formulation further comprises protease inhibitors.

It is possible that other ingredients may be present in the peptide pharmaceutical formulation of the present invention. Such additional ingredients may include wetting agents, emulsifiers, antioxidants, bulking agents, tonicity modifiers, chelating agents, metal ions, oleaginous vehicles, proteins (e.g., human serum albumin, gelatine or proteins) and a zwitterion (e.g., an amino acid such as betaine, taurine, arginine, glycine, lysine and histidine). Such additional ingredients, of course, should not adversely affect the overall stability of the pharmaceutical formulation of the present invention.

Formulations intended for oral use may be prepared according to any known method, and such formulations may contain one or more agents selected from the group consisting of sweetening agents, flavouring agents, colouring agents, and preserving agents in order to provide pharmaceutically elegant and palatable preparations. Tablets may contain the active ingredient in a mixture with non-toxic pharmaceutically-acceptable excipients which are suitable for the manufacture of tablets. These excipients may be for example, inert diluents, such as mannitol, maltodextrin, kaolin, calcium carbonate, sodium carbonate, lactose, calcium phosphate or sodium phosphate; granulating and disintegrating agents, for example corn starch; binding agents, for example, starch, gelatine, polymers or acacia; and lubricating agents, for example magnesium stearate, stearic acid or talc. The tablets may be uncoated or they may be coated by known techniques to delay disintegration or release of the therapeutically active polypeptide.

The orally administerable formulations of the present invention may be prepared and administered according to methods well known in pharmaceutical chemistry, see Remington's Pharmaceutical Sciences, 17^{th} ed. (A. Osol ed., 1985).

In one aspect of the invention, the pharmaceutical compositions of the present invention may be administered by means of solid dosage forms such as tablets and capsules. The tablets may be prepared by wet granulation, by dry granulation, by direct compression or melt granulation.

Tablets for this invention may be prepared utilizing conventional tabletting techniques. A general method of manufacture involves blending of an hybrid analogue, a water-soluble diluent, hydrophilic binder and optionally a portion of a disintegrant. This blend is then granulated with an aqueous solution of the hydrophilic binder or an aqueous solution of the hydrophilic binder and surfactant and milled, if necessary. The granules are dried and reduced to a suitable size. Any other ingredients, such as lubricants, (e.g. magnesium stearate) and additional disintegrants, are added to the granules and mixed. This mixture is then compressed into a suitable size and shape using conventional tabletting machines such as a rotary tablet press. The tablets may be film coated by techniques well known in the art.

Formulations for oral use may also be presented as hard or soft gelatine capsules where the active ingredient is mixed with an inert solid diluent, for example, such as mannitol, maltodextrin, calcium carbonate, sodium carbonate, lactose, kaolin, calcium phosphate or sodium phosphate, or a soft gelatine capsule wherein the active ingredient is mixed with water or an oil medium, for example peanut oil, liquid paraffin, or olive oil.

Capsules for this invention may be prepared utilizing conventional methods. A general method of manufacture involves blending a therapeutically active peptide, alginate, a water-soluble diluent, a hydrophilic binder, and optionally a portion of a disintegrant. This blend is then granulated with an aqueous solution of the hydrophilic binder or an aqueous solution of the hydrophilic binder and surfactant in water, and milled, if necessary. The granules are dried and reduced to a suitable size. Any other ingredients, such as a lubricant, are added to the granules and mixed. The resulting mixture is then filled into a suitable size hard-shell gelatin capsule using conventional capsule-filling machines.

Pharmaceutical compositions containing a derivative of a hybrid peptide according to the present invention may be administered to a patient in need of such treatment at several sites, for example, at topical sites, for example, skin and mucosal sites, at sites which bypass absorption, for example, administration in an artery, in a vein, in the heart, and at sites which involve absorption, for example, administration in the skin, under the skin, in a muscle or in the abdomen.

Administration of pharmaceutical compositions according to the invention may be through several routes of administration, for example, lingual, sublingual, buccal, in the mouth, oral, in the stomach and intestine, nasal, pulmonary, for example, through the bronchioles and alveoli or a combination thereof, epidermal, dermal, transdermal, vaginal, rectal, ocular, for examples through the conjunctiva, uretal, and parenteral to patients in need of such a treatment.

Compositions of the current invention may be administered in several dosage forms, for example, as solutions, suspensions, emulsions, microemulsions, multiple emulsion, foams, salves, pastes, plasters, ointments, tablets, coated tablets, rinses, capsules, for example, hard gelatine capsules and soft gelatine capsules, suppositories, rectal capsules, drops, gels, sprays, powder, aerosols, inhalants, eye drops, ophthalmic ointments, ophthalmic rinses, vaginal pessaries, vaginal rings, vaginal ointments, injection solution, in situ transforming solutions, for example in situ gelling, in situ setting, in situ precipitating, in situ crystallization, infusion solution, and implants.

Compositions of the invention may further be compounded in, or attached to, for example through covalent, hydrophobic and electrostatic interactions, a drug carrier, drug delivery system and advanced drug delivery system in order to further enhance stability of the derivative of a hybrid peptide increase bioavailability, increase solubility, decrease adverse effects, achieve chronotherapy well known to those skilled in the art, and increase patient compliance or any combination thereof.

Compositions of the current invention are useful in the formulation of solids, semisolids, powder and solutions for pulmonary administration of the derivative of human amylin or an analogue thereof, using, for example a metered dose inhaler, dry powder inhaler and a nebulizer, all being devices well known to those skilled in the art.

Compositions of the current invention are useful in the formulation of controlled, sustained, protracting, retarded, and slow release drug delivery systems.

More specifically, but not limited to, compositions may be useful in formulation of parenteral controlled release and sustained release systems (both systems leading to a many-fold reduction in number of administrations), well known to those skilled in the art. Even more preferably, are controlled release and sustained release systems administered subcutaneous. Without limiting the scope of the invention, examples of useful controlled release system and compositions are hydrogels, oleaginous gels, liquid crystals, polymeric micelles, microspheres, nanoparticles.

Methods to produce controlled release systems useful for compositions of the current invention include, but are not limited to, crystallization, condensation, co-crystallization, precipitation, co-precipitation, emulsification, dispersion, high pressure homogenisation, encapsulation, spray drying, microencapsulating, coacervation, phase separation, solvent evaporation to produce microspheres, extrusion and supercritical fluid processes. General reference is made to Handbook of Pharmaceutical Controlled Release (Wise, D. L., ed. Marcel Dekker, New York, 2000) and Drug and the Pharmaceutical Sciences vol. 99: Protein Formulation and Delivery (MacNally, E. J., ed. Marcel Dekker, New York, 2000).

Parenteral administration may be performed by subcutaneous, intramuscular, intraperitoneal or intravenous injection by means of a syringe, optionally a pen-like syringe. Alternatively, parenteral administration can be performed by means of an infusion pump. A further option is a composition which may be a solution or suspension for the administration of the hybrid analogue compound in the form of a nasal or pulmonal spray. As a still further option, the pharmaceutical compositions containing the hybrid analogue compound of the invention can also be adapted to transdermal administration, e.g. by needle-free injection or from a patch, optionally an iontophoretic patch, or transmucosal, e.g. buccal, administration.

The derivative of a hybrid peptide according to the invention can be administered via the pulmonary route in a vehicle, as a solution, suspension or dry powder using any of known types of devices suitable for pulmonary drug delivery. Examples of these comprise of, but are not limited to, the three general types of aerosol-generating for pulmonary drug delivery, and may include jet or ultrasonic nebulizers, metered-dose inhalers, or dry powder inhalers (Cf. Yu J, Chien Y W. Pulmonary drug delivery: Physiologic and mechanistic aspects. Crit Rev Ther Drug Carr Sys 14 (4) (1997) 395-453).

Parenteral administration may be performed by subcutaneous, intramuscular, intraperitoneal or intravenous injection by means of a syringe, optionally a pen-like syringe. Alternatively, parenteral administration can be performed by means of an infusion pump. A further option is a composition which may be a solution or suspension for the administration of the derivative of a hybrid peptide in the form of a nasal or pulmonal spray. As a still further option, the pharmaceutical compositions containing the derivative of a hybrid peptide of the invention can also be adapted to transdermal administration, e.g. by needle-free injection or from a patch, optionally an iontophoretic patch, or transmucosal, e.g. buccal, administration.

In one aspect of the invention the pharmaceutical formulation comprising the derivative of a hybrid peptide is stable for more than 6 weeks of usage and for more than 3 years of storage.

In another aspect of the invention the pharmaceutical formulation comprising the derivative of a hybrid peptide is stable for more than 4 weeks of usage and for more than 3 years of storage.

In a further aspect of the invention the pharmaceutical formulation comprising the derivative of a hybrid peptide is stable for more than 4 weeks of usage and for more than two years of storage.

In an even further aspect of the invention the pharmaceutical formulation comprising the derivative of a hybrid peptide is stable for more than 2 weeks of usage and for more than two years of storage.

Aqueous suspensions may contain the active compounds in admixture with excipients suitable for the manufacture of aqueous suspensions.

Oily suspensions may be formulated by suspending the active ingredient in a vegetable oil, for example arachis oil, olive oil, sesame oil or coconut oil, or in a mineral oil such as a liquid paraffin. The oily suspensions may contain a thickening agent, for example bees-wax, hard paraffin or cetyl alcohol. Sweetening agents such as those set forth above, and flavouring agents may be added to provide a palatable oral preparation. These formulations may be preserved by the addition of an anti-oxidant such as ascorbic acid.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example, sweetening, flavouring, and colouring agents may also be present.

The pharmaceutical formulations comprising a compound for use according to the present invention may also be in the form of oil-in-water emulsions. The oily phase may be a vegetable oil, for example, olive oil or arachis oil, or a mineral oil, for example a liquid paraffin, or a mixture thereof. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, preservative and flavouring and colouring agent.

In a further embodiment of the invention, the formulation further comprises a permeation enhancer. Bile salts and fatty acids are most often considered to increase the oral permeability of the lipid bi-layer membranes of the epithelial cell lining of the GI tract. In general, permeation enhancers increase paracellular and trancellular transport of macropeptides by reversible altering the membrane integrity. The bile salt is selected from the group consisting of cholate, deoxycholate, taurocholate, glycocholate, taurodeoxycholate, ursodeoxycholate, tauroursodeoxycholate, and chenodeoxycholate. The fatty acids is selected from the group of short, medium and long chain fatty acids, such as caprylic acid, capric acid, lauric acid, myristic acid, plamitic acid, stearic acid etc. Others enhancers could be surfactants such as monoglycerides, polyoxyethylene esters, sorbitan surfactants (noninic) and sulphates (anionic).

In a further embodiment of the invention, the formulation further comprises a muco-adhesive polymer. An intimate contact of the drug delivery system to the mucosa of the gastrointestinal tract can be obtained by use of such a mucoadhesive polymer. An intimate contact of the dosage form to the membrane seems advantageous as an enzymatic degradation of the therapeutically active polypeptide on the way between the delivery system and the absorption membrane can be avoided. Moreover, a step concentration gradient on the absorption membrane representing the driving force for passive drug uptake can be provided.

In a further embodiment of the invention, the formulation further comprises an inhibitor of a proteolytic enzyme(s) to further circumvent the enzymatic barrier and achieving the delivery of the therapeutically active polypeptide such as aminopeptidase inhibitor, amastatin, bestatin, boroleucine and puromycin. Examples of protease inhibitors are sodium glycolate, camostat mesilate, bacitracin, soybean trypsin inhibitor and aprotinin.

Entrapment and encapsulation is a technique used in drug delivery systems for therapeutically active polypeptides to optimize delivery properties including protection against enzymatic degradation. Entrapment or encapsulation could be in the form of polymeric drug delivery systems such as hydrogels and nanocapsules/microspheres, and lipid drug delivery systems such as liposomes and micro emulsions.

Formulations of the current invention may be administered in several dosage forms, for example, as solutions, suspensions, micro- and nano suspension, emulsions, microemulsions, multiple emulsion, foams, salves, pastes, ointments, tablets, coated tablets, effervescent tablets, sublingual tablets, buccal tablets, capsules, for example, hard gelatine capsules and soft gelatine capsules, powder, granules, in situ transforming solutions, for example in situ gelling, in situ setting, in situ precipitating, in situ crystallization, stomach floating formulation such as floating suspension, floating tablet or the like.

In another aspect, the present invention relates to a derivative according to the invention for use as a medicament.

In one aspect, a hybrid derivative according to the invention is used for the preparation of a medicament for the treatment or prevention of hyperglycemia, type 2 diabetes, impaired glucose tolerance, type 1 diabetes, obesity, hypertension, syndrome X, dyslipidemia, cognitive disorders, atherosclerosis, myocardial infarction, stroke, coronary heart disease and other cardiovascular disorders, inflammatory bowel syndrome, dyspepsia and gastric ulcers.

In another aspect, a hybrid derivative according to the invention is used as a medicament for delaying or preventing disease progression in type 2 diabetes.

In another aspect, a hybrid derivative according to the invention is used as a medicament for decreasing food intake, decreasing β-cell apoptosis, increasing β-cell function and β-cell mass, and/or for restoring glucose sensitivity to β-cells.

In one aspect of the invention, the hybrid derivative according to the invention is for use as a medicament for the treatment or prevention of hyperglycemia, type 2 diabetes, impaired glucose tolerance, type 1 diabetes, obesity, hypertension, syndrome X, dyslipidemia, cognitive disorders, atherosclerosis, myocardial infarction, coronary heart disease and other cardiovascular disorders, stroke, inflammatory bowel syndrome, dyspepsia and gastric ulcers or for delaying or preventing disease progression in type 2 diabetes or for decreasing food intake, decreasing β-cell apoptosis, increasing β-cell function and β-cell mass, and/or for restoring glucose sensitivity to β-cells, is provided.

In a further aspect of the invention, a method for the treatment or prevention of hyperglycemia, type 2 diabetes, impaired glucose tolerance, type 1 diabetes, obesity, hypertension, syndrome X, dyslipidemia, cognitive disorders, atherosclerosis, myocardial infarction, coronary heart disease and other cardiovascular disorders, stroke, inflammatory bowel syndrome, dyspepsia and gastric ulcers or for delaying or preventing disease progression in type 2 diabetes or for decreasing food intake, decreasing β-cell apoptosis, increasing β-cell function and β-cell mass, and/or for restoring glucose sensitivity to β-cells, the method comprising administering to a patient in need of such treatment an effective amount for such treatment of a hybrid derivative according to the invention, is provided.

The treatment with a hybrid derivative according to the present invention may also be combined with a second or more pharmacologically active substances, e.g. selected from antidiabetic agents, antiobesity agents, appetite regulating agents, antihypertensive agents, agents for the treatment and/or prevention of complications resulting from or associated with diabetes and agents for the treatment and/or prevention of complications and disorders resulting from or associated with obesity. Examples of these pharmacologically active substances are: Insulin, sulphonylureas, biguanides, meglitinides, glucosidase inhibitors, glucagon antagonists, DPP-IV (dipeptidyl peptidase-IV) inhibitors, MC4 inhibitors, liptin, PYY, PP or inhibitors of hepatic enzymes involved in stimulation of gluconeogenesis and/or glycogenolysis, glucose uptake modulators, compounds modifying the lipid metabolism such as antihyperlipidemic agents as HMG CoA inhibitors (statins), compounds lowering food intake, RXR agonists and agents acting on the ATP-dependent potassium channel of the β-cells; Cholestyramine, colestipol, clofibrate, gemfibrozil, lovastatin, pravastatin, simvastatin, probucol, dextrothyroxine, neteglinide, repaglinide; β-blockers such as alprenolol, atenolol, timolol, pindolol, propranolol and metoprolol, ACE (angiotensin converting enzyme) inhibitors such as benazepril, captopril, enalapril, fosinopril, lisinopril, alatriopril, quinapril and ramipril, calcium channel blockers such as nifedipine, felodipine, nicardipine, isradipine, nimodipine, diltiazem and verapamil, and α-blockers such as doxazosin, urapidil, prazosin and terazosin; CART (cocaine amphetamine regulated transcript) agonists, NPY (neuropeptide Y) antagonists, MC4 (melanocortin 4) agonists, orexin antagonists, TNF (tumor necrosis factor) agonists, CRF (corticotropin releasing factor) agonists, CRF BP (corticotropin releasing factor binding protein) antagonists, urocortin agonists, β3 agonists, MSH (melanocyte-stimulating hormone) agonists, MCH (melanocyte-concentrating hormone) antagonists, CCK (cholecystokinin) agonists, serotonin re-uptake inhibitors, serotonin and noradrenaline re-uptake inhibitors, mixed serotonin and noradrenergic compounds, 5HT (serotonin) agonists, bombesin agonists, galanin antagonists, growth hormone, growth hormone releasing compounds, TRH (thyreotropin releasing hormone) agonists, UCP 2 or 3 (uncoupling protein 2 or 3) modulators, leptin agonists, DA agonists (bromocriptin, doprexin), lipase/amylase inhibitors, RXR (retinoid X receptor) modulators, TR β agonists; histamine H3 antagonists, gastrin and gastrin analogs.

It should be understood that any suitable combination of the derivatives according to the invention with one or more of the above-mentioned compounds and optionally one or more further pharmacologically active substances are considered to be within the scope of the present invention.

The following is a non-limiting list of aspects, which is further described elsewhere herein:

1. A derivative of a hybrid peptide, wherein said hybrid peptide comprises the C-terminal end of the human amylin peptide sequence, the middle portion of the salmon calcitonin peptide sequence and the N-terminal end of the human amylin peptide sequence, and wherein an albumin binding moiety is attached to the hybrid peptide, optionally via a linker.

2. A derivative according to aspect 1 which is the parent hybrid peptide (SEQ ID No: 5) or an analogue hybrid peptide, wherein an albumin binding moiety is attached to the hybrid peptide, optionally via a linker.

3. A derivative according to aspect 1 or 2 which is an analogue hybrid peptide, wherein an albumin binding moiety is attached to the hybrid peptide, optionally via a linker.

4. A derivative according to aspect 3, wherein the analogue hybrid peptide has from 1-12 amino acid substitutions compared to the parent hybrid peptide.

5. A derivative according to aspect 3 or 4, wherein the analogue hybrid peptide has from 1-10 amino acid substitutions compared to the parent hybrid peptide.

6. A derivative according to any one of aspects 3-5, wherein the analogue hybrid peptide has from 1-8 amino acid substitutions compared to the parent hybrid peptide.

7. A derivative according to any one of aspects 3-6, wherein the analogue hybrid peptide has from 1-6 amino acid substitutions compared to the parent hybrid peptide.

8. A derivative according to any one of aspects 3-7, wherein the analogue hybrid peptide has from 1-4 amino acid substitutions compared to the parent hybrid peptide.

9. A derivative according to any one of the preceding aspects, which is selected from the group consisting of:
amylin(1-7)-[Arg11,Arg18]sCT(8-27)-amylin(33-37) (seq ID No: 5)
amylin(2-7)-[Arg11,Arg18]sCT(8-27)-amylin(33-37) (seq ID No: 6)
amylin(1-8)-[Arg11,Arg18]sCT(9-27)-amylin(33-37) (seq ID No: 7)
amylin(2-8)-[Arg11,Arg18]sCT(9-27)-amylin(33-37) (seq ID No: 8)
[His1]amylin(1-8)-[His11,His18,His24]sCT(9-27)-amylin (33-37) (seq ID No: 9)
   wherein an albumin binding moiety is attached to the hybrid peptide, optionally via a linker.

10. A derivative according to any one of the preceding aspects wherein the albumin binding moiety is attached to the N-terminal amino acid and/or the C-terminal amino acid and/or to one or more amino acids internally in the hybrid peptide.

11. A derivative according to anyone of the preceding aspects wherein the derivative comprises an N-terminal extension consisting of 1-12 additional amino acids attached N-terminally to the hybrid peptide, wherein the albumin binding moiety is attached, optionally via a linker, to the N-terminal amino acid of the additional amino acids.

12. A derivative according to aspect 11, wherein the N-terminal extension consists of 1-10 amino acids, wherein the albumin binding moiety is attached, optionally via a linker, to the N-terminal amino acid of the additional amino acids.

13. A derivative according to aspect 12, wherein the N-terminal extension consists of 1-8 amino acids, wherein the albumin binding moiety is attached, optionally via a linker, to the N-terminal amino acid of the additional amino acids.

14. A derivative according to aspect 13, wherein the N-terminal extension consists of 1-6 amino acids, wherein the albumin binding moiety is attached, optionally via a linker, to the N-terminal amino acid of the additional amino acids.

15. A derivative according to aspect 14, wherein the N-terminal extension consists of 5 amino acids, wherein the albumin binding moiety is attached, optionally via a linker, to the N-terminal amino acid of the additional amino acids.

16. A derivative according to aspect 15, wherein the N-terminal extension consists of 4 amino acids, wherein the albumin binding moiety is attached, optionally via a linker, to the N-terminal amino acid of the additional amino acids.

17. A derivative according to aspect 16, wherein the N-terminal extension consists of 3 amino acids, wherein the albumin binding moiety is attached, optionally via a linker, to the N-terminal amino acid of the additional amino acids.

18. A derivative according to aspect 17, wherein the N-terminal extension consists of 2 amino acids, wherein the albumin binding moiety is attached, optionally via a linker, to the N-terminal amino acid of the additional amino acids.

19. A derivative according to aspect 18, wherein the N-terminal extension consists of 1 amino acid, wherein the albumin binding moiety is attached, optionally via a linker, to the additional amino acid.

20. A derivative according to any one of the preceding aspects, wherein 0-8 additional charges have been added compared to the parent hybrid peptide.

21. A derivative according to any one of the preceding aspects, wherein 0-8 additional positive charges have been added compared to the parent hybrid peptide.

22. A derivative according to aspect 20, wherein the 0-8 additional charges have been added by substituting one or more amino acid residues of the parent hybrid peptide with charged amino acids and/or by adding charged amino acids in an N-terminal extension, or by adding negatively charged entities in the albumin binding residue and/or the linker.

23. A derivative according to aspect 22, wherein the 0-8 additional charges have been added by substituting one or more amino acid residues of the parent hybrid peptide with histidine residue(s) and/or arginine residue(s) and/or by adding charged amino acids in a N-terminal extention.

24. A derivative according aspect 23, wherein the 0-8 additional charges have been added by adding charged amino acids in a N-terminal extension.

25. A derivative according aspect 24, wherein the 0-8 additional charges have been added by adding histidine and/or arginine amino acids in a N-terminal extension.

26. The derivative according to any one of the preceding aspects, wherein the albumin binding residue is a lipophilic residue.

27. The derivative according to any one of the preceding aspects, wherein the albumin binding residue is negatively charged at physiological pH.

28. The derivative according to any one of the preceding aspects, wherein the albumin binding residue comprises a group which can be negatively charged.

29. The derivative according to aspect 28, wherein the albumin binding residue comprises a carboxylic acid group.

30. The derivative according to any one of the preceding aspects, wherein the albumin binding residue binds non-covalently to albumin.

31. The derivative according to any one of the preceding aspects, wherein the albumin binding residue has a binding affinity towards human serum albumin that is below about 10 μM or below about 1 μM.

32. The derivative according to any one of the preceding aspects, wherein the albumin binding residue is selected from the group consisting of a straight chain alkyl group, a branched alkyl group, a group which has an ω-carboxylic acid group, and a partially or completely hydrogenated cyclopentanophenanthrene skeleton.

33. The derivative according to any one of the preceding aspects, wherein the albumin binding residue is a cibacronyl residue.

34. The derivative according to aspect 26, wherein the lipophilic residue comprises a partially or completely hydrogenated cyclopentanophenathrene skeleton.

35. The derivative according to aspect 26, wherein the albumin binding residue has from 6 to 40 carbon atoms, from 8 to 26 carbon atoms or from 8 to 20 carbon atoms.

36. The derivative according to aspect 26, wherein the albumin binding residue is an acyl group selected from the group comprising CH₃(CH₂)ᵣCO—, wherein r is an integer from 4 to 38, preferably an integer from 4 to 24, more preferred selected from the group comprising CH₃(CH₂)₆CO—, CH₃(CH₂)₈CO—, CH₃(CH₂)₁₀CO—, CH₃(CH₂)₁₂CO—, CH₃(CH₂)₁₄CO—, CH₃(CH₂)₁₆CO—, CH₃(CH₂)₁₈CO—, CH₃(CH₂)₂₀CO— and CH₃(CH₂)₂₂CO—.

37. The derivative according to aspect 26, wherein the albumin binding residue is an acyl group of a straight-chain or branched alkane α,ω-dicarboxylic acid.

38. The derivative according to aspect 26, wherein the albumin binding residue is an acyl group selected from the group comprising HOOC(CH₂),CO—, wherein s is an integer from 4 to 38, preferably an integer from 4 to 24, more preferred selected from the group comprising HOOC(CH₂)₁₄CO—, HOOC(CH₂)₁₆CO—, HOOC(CH₂)₁₈CO—, HOOC(CH₂)₂₀CO— and HOOC(CH₂)₂₂CO—.

39. The derivative according to aspect 26, wherein the albumin binding residue is a group of the formula CH₃(CH₂)_{w}CO—NHCH(COOH)(CH₂)₂CO—, wherein v is an integer from 10 to 24.

40. The derivative according to aspect 26, wherein the albumin binding residue is a group of the formula CH₃(CH₂)_{w}CO—NHCH((CH₂)₂COOH)CO—, wherein w is an integer from 8 to 24.

41. The derivative according to aspect 26, wherein the albumin binding residue is a group of the formula COOH(CH₂)xCO—, wherein x is an integer from 8 to 24.

42. The derivative according to aspect 26, wherein the albumin binding residue is a group of the formula —NHCH(COOH)(CH₂)₄NH—CO(CH₂)_{y}CH₃, wherein y is an integer from 8 to 18.

43. The derivative according to any one of aspects 1-25, wherein the albumin binding residue is a peptide, such as a peptide comprising less than 40 amino acid residues.

44. The derivative according to any one of the preceding aspects, wherein the albumin binding residue optionally via a linker is connected via the ε-amino group of a lysine residue.

45. A pharmaceutical composition comprising a derivative according to any one of the aspects 1-44, and a pharmaceutically acceptable excipient.

46. The pharmaceutical composition according to aspect 45, which is suited for oral or buccal administration.

47. A derivative according to any one of the aspects 1-44 for use as a medicament.

48. A derivative according to any one of the aspects 1-44 for use as a medicament for the treatment or prevention of hyperglycemia, type 2 diabetes, impaired glucose tolerance, type 1 diabetes, obesity, hypertension, syndrome X, dyslipidemia, cognitive disorders, atherosclerosis, myocardial infarction, coronary heart disease and other cardiovascular disorders, stroke, inflammatory bowel syndrome, dyspepsia and gastric ulcers.

49. A derivative according to any one of the aspects 1-44 for use as a medicament for delaying or preventing disease progression in type 2 diabetes.

50. A derivative according to any one of the aspects 1-44 for use as a medicament for decreasing food intake, decreasing β-cell apoptosis, increasing β-cell function and β-cell mass, and/or for restoring glucose sensitivity to β-cells.Use of a derivative according to any one of the aspects 1-42 for the preparation of a medicament.

51. A method for treatment or prevention of hyperglycemia, type 2 diabetes, impaired glucose tolerance, type 1 diabetes, obesity, hypertension, syndrome X, dyslipidemia, cognitive disorders, atherosclerosis, myocardial infarction, coronary heart disease and other cardiovascular disorders, stroke, inflammatory bowel syndrome, dyspepsia and gastric ulcers comprising administering to a subject an active amount of a derivative according to any one of the aspects 1-44.

52. A method for delaying or preventing disease progression in type 2 diabetes comprising administering to a subject an active amount of a derivative according to any one of the aspects 1-44.

53. A method for decreasing food intake, decreasing β-cell apoptosis, increasing β-cell function and β-cell mass, and/or for restoring glucose sensitivity to β-cells comprising administering to a subject an active amount of a derivative according to any one of the aspects 1-44.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law).

All headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The citation and incorporation of patent documents herein is done for convenience only and does not reflect any view of the validity, patentability, and/or enforceability of such patent documents.

This invention includes all modifications and equivalents of the subject matter recited in the claims appended hereto as permitted by applicable law.

### Assays

### Pharmacological Assay (I)

### Experimental Protocol For Efficacy Testing On Appetite With A Hybrid Peptide Or Hybrid Peptide Derivative, Using An Ad Libitum Fed Rat Model

Sprague Dawley (NTac:SD) rats from Taconic Europe, Denmark are used for the experiments. The rats have a body weight 200-250 g at the start of experiment. The rats arrive at least 10-14 days before start of experiment to allow acclimatization to experimental settings. During this period the animals are handled at least 2 times. After arrival rats are housed individually in a reversed light/dark phase (lights off 9:00 am, lights on 9:00 pm), meaning that lights are off during daytime and on during nighttime. Since rats are normally active and eat their major part of their daily food intake during the dark period, rats are dosed in the morning right before lights are turned off. This set-up results in the lowest data variation and highest test sensitivity.

The experiment is conducted in the rats' home cages and rats have free access to food and water througout the acclimatization period and the experiment period. Each dose of derivative is tested in a group of 5-8 rats. A vehicle group of 6-8 rats is included in each set of testing. Rats are dosed once according to body weight with a 0.01-3 mg/kg solution administered intraperitoneally (ip), orally (po) or subcutaneously (sc). The time of dosing is recorded for each group. After dosing, the rats are returned to their home cages, where they then have access to food and water. The food consumption is recorded individually continuously by on-line registration or manually every hour for 7 hours, and then after 24 h and sometimes 48 h. At the end of the experimental session, the animals are euthanised.

The individual data are recorded in Microsoft excel sheets. Outliers are excluded after applying the Grubbs statistical evaluation test for outliers, and the result is presented graphically using the GraphPad Prism program.

### Luciferase Assay (II)

1. Amylin Assay Outline

It has previously been published (Poyner D R et al 2002, Pharmacological Reviews 54 (2) 233-246) that activation of Amylin receptors (coexpression of Calcitonin receptor and receptor activity modifying peptides RAMPs) by Amylin leads to an increase in the intracellular concentration of cAMP. Consequently, transcription is activated at promotors containing multiple copies of the cAMP response element (CRE). It is thus possible to measure Amylin activity by use of a CRE luciferase reporter gene introduced into BHK cells also expressing an Amylin receptor.

2. Construction of An Amylin 3(a)/CRE-Luc Cell Line

A BHK570 cell line stably transfected with the human calcitonin receptor (CTa) and a CRE-responsive luciferase reportergene. The cell line was further transfected with RAMP-3, using standard methods. This turns the Calcitonin receptor into an Amylin 3(a) receptor. Methotrexate, Neomycin, and Hygromycin are selection markers for luciferase, the Calcitonin receptor, and RAMP-3, respectively.

3. Amylin Luciferase Assay

To perform activity assays, BHK Amylin 3(a)//CRE-luc cells were seeded in white 96 well culture plates at a density of about 20.000 cells/well. The cells were in 100 μl growth medium (DMEM with 10% FBS, 1% Pen/Strep, 1 mM Na-pyruvate, 250 nM Methotrexate, 500 μ/ml Neomycin, and 400 μ/ml Hygromycin). After incubation overnight at 37° C. and 5% CO₂, the growth medium was replaced by 50 μl/well assay medium (DMEM (without phenol red), Glumamax™, 10% FBS, and 10 mM Hepes, pH 7,4). Further, 50 μl/well of standard or sample in assay buffer were added. After 4 hours incubation at 37° C. and 5% CO₂, the assay medium with standard or sample were removed and replaced by 100 μl/well PBS. Further, 100 μl/well LucLite™ was added. The plates were sealed and incubated at room temperature for 30 minutes. Finally, luminescence was measured on a TopCounter (Packard) in SPC (single photon counting) mode.

### Assay (III) General Introduction To ThT Fibrillation Assays For the Assessment of Physical Stability of Protein Formulations

Low physical stability of a peptide may lead to amyloid fibril formation, which is observed as well-ordered, thread-like macromolecular structures in the sample eventually resulting in gel formation. This has traditionally been measured by visual inspection of the sample. However, that kind of measurement is very subjective and depending on the observer. Therefore, the application of a small molecule indicator probe is much more advantageous. Thioflavin T (ThT) is such a probe and has a distinct fluorescence signature when binding to fibrils [Naiki et al. (1989) Anal. Biochem. 177, 244-249; LeVine (1999) Methods. Enzymol. 309, 274-284].

The time course for fibril formation can be described by a sigmoidal curve with the following expression [Nielsen et al. (2001) Biochemistry 40, 6036-6046]:

$$F = {f}_{i} + {m}_{i} t + \frac{{f}_{f} + {m}_{f} t}{1 + {e}^{- [ ( t - {t}_{0} ) / τ ]}} \qquad Eq . ( 1 )$$

Here, F is the ThT fluorescence at the time t. The constant t₀ is the time needed to reach 50% of maximum fluorescence. The two important parameters describing fibril formation are the lag-time calculated by t₀−2τ and the apparent rate constant kₐₚₚ=1/τ.

Formation of a partially folded intermediate of the peptide is suggested as a general initiating mechanism for fibrillation. Few of those intermediates nucleate to form a template onto which further intermediates may assembly and the fibrillation proceeds. The lag-time corresponds to the interval in which the critical mass of nucleus is built up and the apparent rate constant is the rate with which the fibril itself is formed.

### Sample Preparation

Samples were prepared freshly before each assay. Each sample composition is described in each example. The pH of the sample was adjusted to the desired value using appropriate amounts of concentrated NaOH and HClO₄ or HCl. Thioflavin T was added to the samples from a stock solution in H₂O to a final concentration of 1 μM.

Sample aliquots of 200 μl were placed in a 96 well microtiter plate (Packard Opti-Plate™-96, white polystyrene). Usually, four or eight replica of each sample (corresponding to one test condition) were placed in one column of wells. The plate was sealed with Scotch Pad (Qiagen).

### Incubation And Fluorescence Measurement

Incubation at given temperature, shaking and measurement of the ThT fluorescence emission were done in a Fluoroskan Ascent FL fluorescence platereader or Varioskan platereader (Thermo Labsystems). The temperature was adjusted to 37° C. The orbital shaking was adjusted to 960 rpm with an amplitude of 1 mm in all the presented data. Fluorescence measurement was done using excitation through a 444 nm filter and measurement of emission through a 485 nm filter.

Each run was initiated by incubating the plate at the assay temperature for 10 min. The plate was measured every 20 minutes for a desired period of time. Between each measurement, the plate was shaken and heated as described.

### Data Handling

The measurement points were saved in Microsoft Excel format for further processing and curve drawing and fitting was performed using GraphPad Prism. The background emission from ThT in the absence of fibrils was negligible. The data points are typically a mean of four or eight samples and shown with standard deviation error bars. Only data obtained in the same experiment (i.e. samples on the same plate) are presented in the same graph ensuring a relative measure of fibrillation between experiments.

The data set may be fitted to Eq. (1). However, since full sigmodial curves in this case are not always achieved during the measurement time, the degree of fibrillation is expressed as ThT fluorescence tabulated as the mean of the samples and shown with the standard deviation at various time points.

### Assay (IV) PK—Determination of T½ In Mini-Pig

T½ is the terminal half-life=In2/λ_{z}, of a compound in plasma. λ_{z} is the first order rate constant associated with the terminal (log-linear) portion of the plasma concentration-time curve and is estimated by linear regression of time vs. log concentration.

T½ values of the calcitonin derivative of the invention is determined by pharmacokinetic studies in male Göttingen mini-pigs from Ellegaard Göttingen Minipigs ApS and the principles of laboratory animal care are followed.

An acclimatisation period of approximately 6-10 days was allowed before the animals entered the study. At start of the acclimatisation period the mini-pigs were about 5 to 12 months old and in the weight range of 7-35 kg. The mini-pigs had two central venous catheters inserted which were used for blood sampling.

The studies were conducted in an animal room which was illuminated to give a cycle of approx 12 hours light and 12 hours darkness. The animals were housed individually.

The animals had free access to domestic quality drinking water during the study, but were typically fasted from overnight before dosing until approx 6-12 hours after dosing. The animals were weighed on arrival and on the days of dosing.

In the present studies the test substances were administered subcutaneously in approx 2 nmol/kg dose. The animals received a single subcutaneous injection. The subcutaneous injection was given on the right side of the neck, approximately 5-7 cm from the ear and 7-9 cm from the middle of the neck. The injections were given with a stopper on the needle, allowing approx 0.5 cm of the needle to be introduced. Each test substance was given to typically three but in some cases two or four animals.

A full plasma concentration-time profile, employing 12-16 sampling points, was obtained from each animal. In example blood samples were collected according to the following schedule:

After subcutaneous administration:

Predose (0), 0.5, 1, 2, 4, 6, 8, 12, 24, 48, 72, 96, 120, 168 and 240 hours after injection. In some cases also additional blood samples up to 288 hours post injection were taken.

At each sampling time, 0.5 to 2 ml of blood was drawn from each animal. The blood samples were taken via the central venous catheter.

The blood samples were collected into EDTA test tubes (i.e. Sarstedt Micro tube 1.3 mL K3E). Blood samples were kept on ice for max 20 min. before centrifugation. Plasma was separated using centrifugation (i.e. at 4° C., 10 min., 1500 G) and was immediately transferred to Micronic tubes. Approximately 200 μl plasma was transferred to each Micronic tube. The plasma was stored at −20° C. until assayed. The plasma samples were assayed for the content of amylin using an ELISA assay.

The plasma concentration-time profiles were analysed by a non-compartmental pharmacokinetic analysis (NCA) using WinNonlin Professional 5.0 (Pharsight Inc., Mountain View, Calif., USA). NCA was performed using the individual plasma concentration-time profiles from each animal. T½ is the terminal half-life=In2/λ_{z}, and was determined from λ_{z}, the first order rate constant associated with the terminal (log-linear) portion of the curve, estimated by linear regression of time vs. log concentration.

### Assay (V) PK—Determination of T½ In Rat

T½ is the terminal half-life=In2/λ_{z} of a compound in plasma. λ_{z} is the first order rate constant associated with the terminal (log-linear) portion of the plasma concentration-time curve and is estimated by linear regression of time vs. log concentration.

T½ values of the calcitonin derivatives of the invention is determined by pharmacokinetic studies in Sprague Dawley male rats, from Taconic Europe and the principles of laboratory animal care are followed.

An acclimatisation period of approximately 7 days was allowed before the animals entered the study. At start of the acclimatisation period the rats were in the weight range of 300-400 g. The rats had permanent catheters inserted in a. carotis which were used for blood sampling.

The studies were conducted in an animal room which was illuminated to give a cycle of approx 12 hours light and 12 hours darkness. The animals were housed individually due to the catheters and had food and water ad lib. The animals were weighed on the days of dosing.

In the present studies the test substances were administered subcutaneously in approx 20 nmol/kg dose. The animals received a single subcutaneous injection to the neck using a 25 G needle with syringe. Each test substance was given to typically three but in some cases two or four animals.

A full plasma concentration-time profile, employing 8-10 sampling points, was obtained from each animal. In example blood samples were collected according to the following schedule:

After subcutaneous administration:

Predose (0), 0.5, 1, 1.5, 2, 4, 6, 12, 24, 48 and 72 hours after injection. At each sampling time, 0.08 to 0.10 ml of blood was drawn from each animal. The blood samples were taken via the catheter.

The blood samples were collected into EDTA test tubes. Blood samples were kept on ice for max 20 min. before centrifugation. Plasma was separated using centrifugation (i.e. at 4° C., 10 min., 1500 G) and was immediately transferred to Micronic tubes or PCR plates. Approximately 40 μl plasma was transferred and was stored at −20° C. until assayed. The plasma samples were assayed for the content of amylin using an ELISA assay.

The plasma concentration-time profiles were analysed by a non-compartmental pharmacokinetic analysis (NCA) using WinNonlin Professional 5.0 (Pharsight Inc., Mountain View, Calif., USA). NCA was performed using the individual plasma concentration-time profiles from each animal. T½ is the terminal half-life=In2/λ_{z} and was determined from λ_{z}, the first order rate constant associated with the terminal (log-linear) portion of the curve, estimated by linear regression of time vs. log concentration.

### Assay (VI)—Determination of Solubility

Solubility versus pH curves were measured in the following way. A formulation was prepared and aliquotes were adjusted to pH values in the desired range by adding HClO₄ or HCl and NaOH. These samples were left equilibrating at room temperature for 2-3 days. Then the samples were centrifuged. A small aliquot of each sample was withdrawn for reverse HPLC analysis for determination of the concentration of the proteins in solution. The pH of each sample was measured after the centrifugation, and the concentration of each protein was depicted versus the measured pH.

### Assay (VII)—Determination of Albumin Binding Affinity

“Albumin binding affinity” may be determined by several methods known within the art. In one method the derivative to be measured is radiolabeled with e.g. ¹²⁵I or ³H and incubated with immobilized albumin (Kurtzhals et.al., Biochem.J., 312, 725-731 (1995)). The binding of the derivative relative to a standard is calculated. In another method a related compound is radio-labeled and its binding to albumin immobilized on e.g. SPA beads is competed by a dilution series of the derivative to be measured. The EC50 value for the competition is a measure of the affinity of the derivative. In a third method, the receptor affinity or potency of a derivative is measured at different concentrations of albumin, and the shift in relative affinity or potency of the derivative as a function of albumin concentration reflects its affinity for albumin.

### EXAMPLES

### Synthesis And Purification

The compounds of the examples were prepared according to the below-mentioned peptide Synthesis:

One method of peptide synthesis was by Fmoc chemistry on a microwave-based Liberty peptide synthesizer (CEM Corp., North Carolina). The resin was Tentagel S RAM with a loading of 0.25 mmol/g. The coupling chemistry was DIC/HOAt in NMP using amino acid solutions of 0.3 M in NMP and a molar excess of 6-8 fold. Coupling conditions was 5 minutes at up to 70° C. Deprotection was with 5% piperidine in NMP at up to 70° C. The protected amino acids used were standard Fmoc-amino acids (supplied from e.g. Anaspec or Novabiochem) dissolved at 0.3 M in NMP containing 0.3 M HOAt.

Another method of peptide synthesis was on an Applied Biosystems 433 peptide synthesizer in 0.25 mmol or 1.0 mmol scale using the manufacturer supplied FastMoc UV protocols which employ HBTU (2-(1H-Benzotriazol-1-yl-)-1,1,3,3 tetramethyluronium hexafluorophosphate) or HATU (O-(7-azabenzotriazol-1-yl)-1,1,3,3-tetramethyluronium hexafluorophosphate) mediated couplings in NMP, and UV monitoring of the deprotection of the Fmoc protection group. The starting resin used for the synthesis of the peptide amides was Rink-Amide resin. The protected amino acid derivatives used were standard Fmoc-amino acids (supplied from e.g. Anaspec, or Novabiochem) supplied in preweighed cartridges suitable for the AB1433A synthesizer.

When a chemical modification of a lysine side chain was desired, the lysine was incorporated as Lys(Mtt) and the N-terminal amino acid was either incorporated into the sequence as a Boc-amino acid or, if the N-terminal amino acid was incorporated as an Fmoc-amino acid, the Fmoc group was removed and the N-terminal was protected by treatment with 6 equivalents of Boc-carbonate and 6 equivalents of DIPEA in NMP for 30 minutes. The resin was washed with NMP and DCM and the Mtt group was removed by suspending the resin in neat hexafluoroisopropanol for 20 minutes followed by washing with DCM and NMP. The chemical modification of the lysine was performed by adding one or more of the building blocks listed below by the same methods as used for the peptide synthesis, i.e. by one or more automated steps on the Liberty or the ABI 433 or by one or more manual coupling steps at room temperature. After synthesis the resin was washed with DCM and dried, and the peptide was cleaved from the resin by a 2 hour treatment with TFA/TIPS/water (92.5/5/2.5) followed by precipitation with 4 volumes of diethylether. After further washing with diethylether and drying, the peptide was redissolved in water at 1-2 mg/ml, pH adjusted to about 4.5, and the disulfide bridge formed by treatment with 1.1 eq. of [Pt(IV)ethylenediamine₂Cl₂]Cl₂ overnight. Alternatively, the disulfide bridge was formed on the resin by treatment with 10 equivalents of iodine in NMP for 1 hour. In this case the crude peptide was purified directly after cleavage and diethylether precipitation.
Purification: The crude peptide was purified by semipreparative HPLC on a 20 mm×250 mm column packed with either 5μ or 7μ C-18 silica. Peptide solutions were pumped onto the HPLC column and precipitated peptides were dissolved in 5 ml 50% acetic acid H₂O and diluted to 20 ml with H₂O and injected on the column which then was eluted with a gradient of 40-60% CH₃CN in 0.1% TFA 10 ml/min during 50 min at 40° C. The peptide containing fractions were collected. The purified peptide was lyophilized after dilution of the eluate with water.
For analysis of HPLC-fractions and final product RP-HPLC analysis was performed using UV detection at 214 nm and e.g. a Vydac 218TP54 4.6mm×250mm 5μ C-18 silica column (The Separations Group, Hesperia, USA) and eluted at e.g. 1 ml/min at 42° C. Most often one of four different elution conditions was used:
A1: Equilibration of the column with a buffer consisting of 0.1M (NH₄)₂SO₄, which was adjusted to pH 2.5 with concentrated H₂SO₄ and elution by a gradient of 0% to 60% CH₃CN in the same buffer during 50 min.
B1: Equilibration of the column with 0.1% TFA/H₂O and elution by a gradient of 0% CH₃CN/0.1% TFA/H₂O to 60% CH₃CN/0.1% TFA/H₂O during 50 min.
B6: Equilibration of the column with 0.1% TFA/H₂O and elution by a gradient of 0% CH₃CN/0.1% TFA/H₂O to 90% CH₃CN/0.1% TFA/H₂O during 50 min.
   Alternatively the RP-HPLC analysis was performed using UV detection at 214 nm and a Symmetry300, 3.6mm×150mm, 3.5μ C-18 silica column (Waters) which was eluted at 1 ml/min at 42° C.
   B4: Equilibration of the column with 0.05% TFA/H₂O and elution by a gradient of 5% CH₃CN/0.05% TFA/H₂O to 95% CH₃CN/0.05% TFA/H₂O during 15 min.
   The identity of the peptide was confirmed by MALDI-MS on a Bruker Microflex.

### Abbreviations Used

HBTU: 2-(1H-Benzotriazol-1-yl-)-1,1,3,3 tetramethyluronium hexafluorophosphate
Fmoc: 9 H-fluoren-9-ylmethoxycarbonyl
Boc: tert butyloxycarbonyl
Mtt: 4-methyltrityl
DCM: dichloromethane
TIPS: triisopropylsilane
TFA: trifluoroacetic acid
NMP: 1-Methyl-pyrrolidin-2-one
HOAt: 1-Hydroxy-7-azabenzotriazole
DIC: Diisopropylcarbodiimide
Trt: triphenylmethyl
   The systematic name of all examples is defined as explained in the description.

### Example 1

### Amylin (1-7)-[Arg11,Arg18]sCT (8-27)-amylin (33-37)

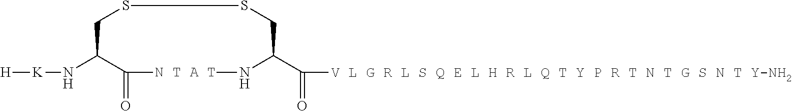

### Example 2

### N-epsilon1-(4—Carboxy-4-(19-carboxy-nonadecanoylamino)butyryl)(amylin (1-7)-[Arg11,Arg18]sCT (8-27)-amylin (33-37)

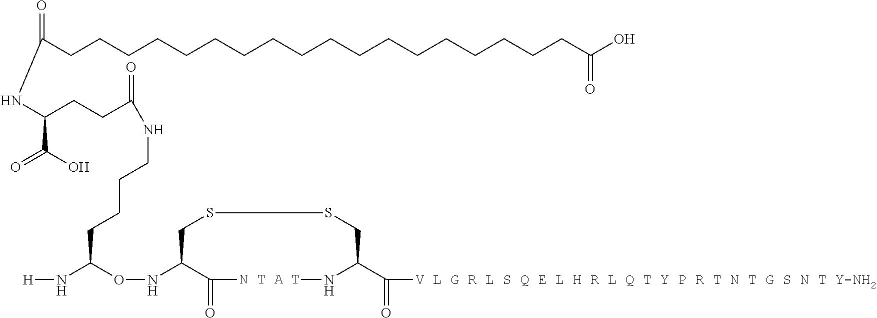

### Example 3

### N-alpha2-(4—Carboxy-4-(19-carboxy-nonadecanoylamino)butyryl)-amylin (2-7)-[Arg11,Arg18]sCT (8-27)-amylin (33-37)

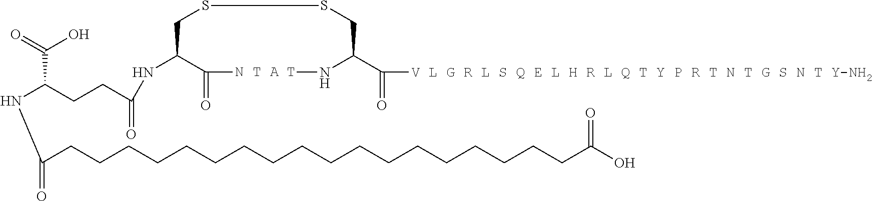

### Example 4

### Amylin (1-8)-[Arg11,Arg18]sCT (9-27)-amylin (33-37)

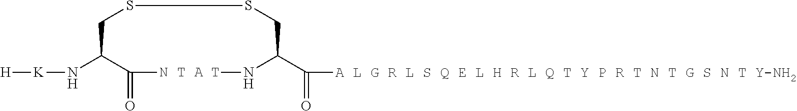

### Example 5

### [His1]amylin (1-8)-[His11,His18,His24]sCT (9-27)-amylin (33-37)

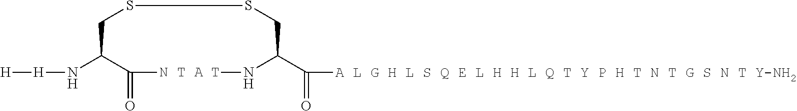

### Example 6

N-alpha1-(4—Carboxy-4-(19-carboxy-nonadecanoylamino)butyryl)-amylin (1-8)-[Arg11,Arg18]sCT (9-27)-amylin (33-37)

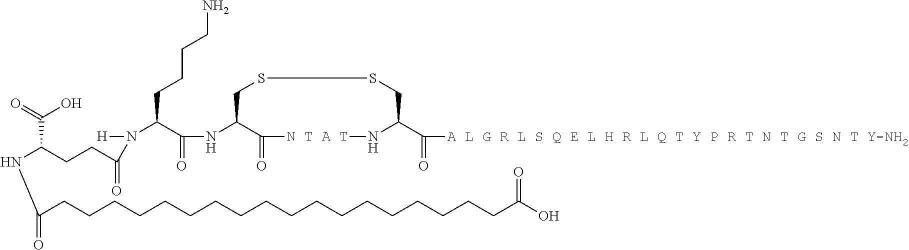

### Potency In Luciferase Assay (II)

| Peptide | EC50 (pM) in luciferase assay |
|---|---|
| Example 1 | 3 |
| Example 2 | 30 |
| Example 3 | 60 |
| Example 4 | 3 |
| Example 5 | 10 |
| Example 6 | 50 |

### Potency In Pharmacological Assay (I)

The table shows the reduction in food intake over a period of 0-24 hours and 24-48 hours. It is seen from the table that the derivatised hybrid peptides (Examples 2, 3 and 6) reduces food intake more effectively than the non-derivatised hybrid peptide (example 1). n=5-7, data=mean, compound dosis: 30 mmol/kg.

| | Reduction in food intake | Reduction in food intake |
|---|---|---|
| Peptide | 0-24 h | 24-48 h |
| Example 1 | 52% | 7% |
| Example 2 | 93% | 98% |
| Example 3 | 88% | 71% |
| Example 6 | 91% | 100% |

1. A derivative of a hybrid peptide, wherein said hybrid peptide comprises the C-terminal end of the human amylin peptide sequence, the middle portion of the salmon calcitonin peptide sequence and the N-terminal end of the human amylin peptide sequence, and wherein an albumin binding moiety is attached to the hybrid peptide, optionally via a linker.

2. A derivative according to claim 1 which is the parent hybrid peptide (SEQ ID No: 5) or an analogue hybrid peptide, wherein an albumin binding moiety is attached to the hybrid peptide, optionally via a linker.

3. A derivative according to claim 2, wherein the analogue hybrid peptide has from 1-12 amino acid substitutions compared to the parent hybrid peptide or where the analogue hybrid peptide is selected from the group consisting of:
amylin(2-7)-[Arg11,Arg18]sCT(8-27)-amylin(33-37) (seq ID No: 6)
amylin(1-8)-[Arg11,Arg18]sCT(9-27)-amylin(33-37) (seq ID No: 7)
amylin(2-8)-[Arg11,Arg18]sCT(9-27)-amylin(33-37) (seq ID No: 8)
[His1]amylin(1-8)-[His11,His18,His24]sCT(9-27)-amylin (33-37) (seq ID No: 9)

4. A derivative according to claim 1 wherein the albumin binding moiety is attached to the N-terminal amino acid and/or the C-terminal amino acid and/or to one or more amino acids internally in the hybrid peptide.

5. A derivative according to claim 1 wherein the derivative comprises an N-terminal extension consisting of 1-12 additional amino acids attached N-terminally to the hybrid peptide, wherein the albumin binding moiety is attached, optionally via a linker, to the N-terminal amino acid of the additional amino acids.

6. A derivative according to claim 1, wherein 0-8 additional charges have been added compared to the parent hybrid peptide.

7. The derivative according to claim 1, wherein the albumin binding residue is a lipophilic residue.

8. The derivative according to claim 7, wherein the albumin binding residue is selected from the group consisting of a straight chain alkyl group, a branched alkyl group, a group which has an co-carboxylic acid group, and a partially or completely hydrogenated cyclopentanophenanthrene skeleton.

9. The derivative according to claim 1, wherein the albumin binding residue optionally via a linker is connected via the ε-amino group of a lysine residue.

10. A pharmaceutical composition comprising a derivative according to claim 1, and a pharmaceutically acceptable excipient.

11-13. (canceled)

14. A method for treating hyperglycemia, type 2 diabetes, impaired glucose tolerance, type 1 diabetes, obesity, hypertension, syndrome X, dyslipidemia, cognitive disorders, atherosclerosis, myocardial infarction, coronary heart disease and other cardiovascular disorders, stroke, inflammatory bowel syndrome, dyspepsia and gastric ulcers, comprising administering to a subject in need of such treatment a therapeutically effective amount of a pharmaceutical composition according to claim 1.

15. A method for decreasing food intake, decreasing β-cell apoptosis, increasing β-cell function and β-cell mass, and/or for restoring glucose sensitivity to β-cells in a subject, the method comprising administering to a subject an effective amount of a pharmaceutical composition according to claim 1.
